(19) United States
(12) Patent Application Publication
(10) Pub. No.: US 2013/0202619 A1
(43) Pub. Date: Aug. 8, 2013

(54) METHODS AND MATERIALS FOR REDUCING LIVER FIBROSIS

(71) Applicant: Michael R. Charlton, Rochester, MN (US)

(72) Inventor: Michael R. Charlton, Rochester, MN (US)

(73) Assignee: MAYO FOUNDATION FOR MEDICAL EDUCATION AND RESEARCH, Rochester, MN (US)

(21) Appl. No.: 13/643,042

(22) PCT Filed: Apr. 22, 2011
(86) PCT No.: PCT/US11/33623

§ 371 Date: Oct. 23, 2012

Related U.S. Application Data
(60) Provisional application No. 61/327,565, filed on Apr. 23, 2010.

(51) Int. Cl. A61K 39/395; C12Q 1/68; G01N 33/566; A61K 31/7088; A61P 1/16

(52) U.S. Cl. 424/172.1; 514/44 A; 435/7.92; 435/6.13

(57) ABSTRACT

This document relates to methods and materials for treating diseases or disorders that are caused by or associated with lumican deposition (e.g., liver fibrosis). For example, methods and materials for reducing liver fibrosis by reducing lumican expression or activity within a mammal (e.g., a human) are provided.

### CROSS-REFERENCE TO RELATED APPLICATIONS

This application claims the benefit of U.S. Provisional Application Ser. No. 61/327,565, filed Apr. 23, 2010. The disclosure of the prior application is considered part of (and is incorporated by reference in) the disclosure of this application.

### STATEMENT AS TO FEDERALLY SPONSORED RESEARCH

Funding for the work described herein was provided by the federal government under grant numbers R01 DK 069757-05 awarded by National Institute of Diabetes and Digestive and Kidney Diseases. The federal government has certain rights in the invention.

### BACKGROUND

1. Technical Field

This document relates to methods and materials for treating diseases or disorders that are caused by or associated with lumican deposition (e.g., liver fibrosis). For example, this document provides methods and materials for reducing liver fibrosis by reducing lumican expression or activity within a mammal (e.g., a human).

2. Background Information

Progressive fibrotic diseases of the liver are a major cause of death throughout the world. Fibrosis is the abnormal accumulation of fibrous tissue that can occur as a part of the wound-healing process in damaged tissue. Liver (hepatic) fibrosis, for example, can occur as part of the wound-healing response to chronic liver injury. Liver fibrosis can be characterized by the accumulation of extracellular matrix that can be distinguished qualitatively from that in normal liver. Left unchecked, hepatic fibrosis can progress to cirrhosis (defined by the presence of encapsulated nodules), liver cancer, liver failure, and death.

### SUMMARY

This document provides methods and materials for treating diseases or disorders that are caused by or associated with lumican deposition (e.g., liver fibrosis). For example, this document provides methods and materials for reducing liver fibrosis by reducing lumican expression or activity within a mammal (e.g., a human). This document also provides methods and materials for determining whether or not a test compound reduces lumican polypeptide expression or activity. In addition, this document provides methods and materials for assessing a profibrotic state in a mammal (e.g., a human) with a liver disorder, based on the presence or absence of lumican polypeptide deposition in the mammal

As described herein, lumican is involved in the development of fibrosis in liver tissue as in vivo administration of CC14 to mice resulted in increased lumican deposition and fibrosis in liver tissue of wild type mice versus no lumican deposition and fibrosis in liver tissue of lumican knockout mice. In addition, lumican polypeptide expression is differentially expressed in an hepatocyte cell line (HuH7), a stellate cell line (LX2), and a chollangiocyte cell line (H69) and can be modulated via treatment with TGFβ1 polypeptides and saturated free fatty acids (FFA).

In general, one aspect of this document features a method for treating a mammal having a liver fibrosis condition. The method comprises, or consists essentially of, administering to the mammal, an inhibitor of lumican under conditions wherein the severity of said liver fibrosis condition is reduced. The mammal can be a human. The method can comprise identifying the mammal as having said liver fibrosis condition prior to the administering step. The method can comprise identifying the mammal as having a liver fibrosis condition and as being in need of administration of an inhibitor of lumican under conditions wherein the severity of said liver fibrosis condition is reduced. The method can comprise assessing the mammal, after the administering step, for a reduction in the severity of liver fibrosis. The inhibitor can be an anti-lumican antibody. The inhibitor can be an siRNA directed against a nucleic acid encoding a lumican polypeptide.

In another aspect, this document features a method for identifying a treatment agent for treating a liver fibrosis condition. The method comprises, or consists essentially of, (a) determining whether or not a test agent inhibits lumican expression or activity, wherein inhibition of lumican expression or activity indicates that the test agent is a candidate agent, and (b) administering the candidate agent to a mammal having a liver fibrosis condition to determine whether or not the candidate agent reduces the severity of the liver fibrosis condition, wherein a reduction in the severity indicates that the candidate agent is the treatment agent. Step (a) can comprise using an in vitro lumican expression assay. The mammal can be a mouse.

Another aspect of this document features a method for treating a mammal suspected to develop a liver fibrosis condition. The method comprises, or consists essentially of, administering, to the mammal, an inhibitor of lumican under conditions wherein development of a liver fibrosis condition is reduced. The mammal can be a human. The method can comprise identifying a mammal as being likely to develop a liver fibrosis condition prior to an administering step. The method can comprise identifying a mammal as being likely to develop a liver fibrosis condition and as being in need of the administration. The method can comprise assessing a mammal, after the administering step, for a reduction in the development of a liver fibrosis condition. The inhibitor can be an anti-lumican antibody. The inhibitor can be an siRNA directed against a nucleic acid encoding a lumican polypeptide.

In another aspect, this document features a method of identifying a mammal in need of treatment with an inhibitor of lumican. The method comprises, or consists essentially of, detecting the presence of an elevated level of lumican polypeptide in a mammal, and classifying a mammal as being in need of treatment with an inhibitor of lumican based at least in part on the presence of the elevated level. The mammal can be a human. The mammal can be a mammal suspected of having liver fibrosis.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A and 1B are photographs of liver sections of C57B1/6 mice fed standard rodent chow (n=7) or ‘fast food’, a diet rich in saturated fatty acids and cholesterol (n=8) for a period of 6 months that were stained for lumican, an ECM proteoglycan. All mice were also provided fructose and glucose in drinking water. FIG. 1C is a graph plotting digital image analysis data of lumican staining. FIG. 1D is a graph plotting lumican gene expression data. These results show significantly increased expression of lumican in mice provided the fast food diet. FIGS. 1E and 1F demonstrate that lumican gene expression is positively correlated with TGFβ1 expression (FIG. 1E) and collagen expression (FIG. 1F) in animals fed the standard chow and fast food diets.

FIG. 2 contains graphs showing measurement of serum AST levels in lumican −/−, lumican +/−, and lumican +/+ mice treated with CC14 at different time points by kinetic ultraviolet method.

FIG. 3 contains photographs of an immunohistochemistry analysis that was used to examine lumican expression in liver tissue of lumican −/−, lumican +/−, and lumican +/+ mice treated with CC14 at different time points.

FIG. 4A is a graph plotting gene expression of CYP2E1, which converts CC14 to its toxic form, CC13, and was similar between lumican −/− and lumican +/+ mice. FIG. 4B contains photographs of liver sections of lumican −/− or lumican +/+ administered CC14 or vehicle and stained for TUNEL. FIG. 4C is a graph plotting the level of apoptosis in lumican −/− mice administered CC14 as compared to their wild type counterparts. Apoptosis was minimal in animals administered vehicle alone. FIG. 4D is a graph plotting TNFα gene expression by lumican −/− mice administered CC14 as compared to the lumican +/+ animals.

FIG. 5 contains graphs plotting expression levels of TNFα, IL-1β, and IL-6 in liver lysates of lumican −/− and lumican +/+ animals administered CC14 or vehicle alone. There was a marginally increased expression of TNFα in lumican −/− animals (p=0.09); no difference in IL-1β, and decreased expression of IL-6 in lumican −/− animals administered CC14.

FIG. 6 contains graphs of an histological analysis of lumican polypeptide expression in liver tissue of lumican −/−, lumican +/−, and lumican +/+ mice treated with CC14 at different time points.

FIG. 7 is a graph plotting lumican gene expression in liver tissue of lumican −/−, lumican +/−, and lumican +/+ mice treated for one month with CC14.

FIG. 8 contains photographs of HE staining in liver tissue of lumican −/−, lumican +/−, and lumican +/+ mice treated with CC14 at different time points. These results demonstrate a necro-inflammatory response in all animals receiving CC14.

FIG. 9 contains photographs of Masson's trichrome staining that was performed on liver tissue from of lumican −/−, lumican +/−, and lumican +/+ mice treated with CC14 at different time points. This figure demonstrates near complete lack of stainable fibrous tissue in lumican −/− animals. Fibrous tissue is increased in the lumican +/− and +/+ animals.

FIG. 10 contains photographs in grayscale for collagen stained with picrosirius red performed on liver tissue from of lumican −/−, lumican +/− and lumican +/+ mice treated with CC14 at different time points. Similar to Masson's trichrome, picrosirius red staining demonstrates near complete lack of stainable fibrosis in lumican −/− animals. Fibrosis is markedly increased in the lumican +/− and +/+ animals.

FIG. 11 contains grayscale photographs of picrosirius red staining for detection of collagen expression that was performed on liver tissue from lumican −/−, lumican +/−, and lumican +/+ mice treated with CC14 at different time points.

FIG. 12 is a graph plotting results from an histological analysis of collagen protein abundance in liver tissue from lumican −/−, lumican +/− and lumican +/+ mice treated with CC14 at the three month time point, expressed as percentage of biopsy area. Collagen abundance in lumican −/− animals after three months of CC14 administration is similar to that of animals who received vehicle (no CC14). Collagen is markedly increased in the lumican +/− and lumican +/+ animals.

FIG. 13A contains results from a gene expression analysis for collagen 1a1, one of the principal collagen fibril types associated with fibrosis. The results show that collagen expression was similar between the lumican −/− and the lumican +/+ animals. FIG. 13B contains photographs of liver sections of lumican −/− mice and lumican +/+ animals administered CC14 or vehicle alone and stained for alpha smooth muscle actin (ASMA), a marker for hepatic stellate cell activation. Digital image analysis indicated an increased ASMA expression in lumican −/− animals. TGFβ1 expression was significantly increased in lumican −/− as compared to lumican +/+ animals (FIG. 13C). FIG. 13D is a graph plotting TGFβ1 expression. FIG. 13E contains ultrastructural micrographs (Magnification: 80,000×) of normal lumican −/− and lumican +/+ animals showing that collagen fibrils are of uneven diameter and are scattered in the lumican −/− animals. In lumican +/+ animals, collagen fibrils are of uniform diameter and are packed uniformly. FIG. 13F is a graph plotting MMP13 expression.

FIG. 14 is a photograph of expression levels of fibromodulin in the indicated animals.

FIG. 15 contains photographs of liver tissue from lumican −/− (top row), lumican +/− (middle row), and lumican +/+ (bottom row) mice treated with CC14 at different time points and stained for alpha smooth muscle actin, indicating no effect of lumican on hepatic stellate cell activation.

FIG. 16 contains photographs and a graph of an immunohistochemical staining analysis for Ki67, a marker of cell proliferation. These results demonstrate increased proliferative response of hepatocytes in lumican −/− and lumican +/− animals when compared to lumican +/+ animals.

FIG. 17 contains photographs of staining of liver tissue from lumican −/−, lumican +/−, and lumican +/+ mice treated with CC14 at different time points. Samples were stained with anti-smooth muscle actin and anti-lumican. ASMA staining is apparent in the same anatomical distribution as lumican staining, corresponding to the portal tracts and zone 3.

FIG. 18 contains photographs of immunohistochemical staining for lumican in C57B1/6 mice reared on a standard chow as compared with serial sections of IHC for lumican and ASMA in mice reared on “FF”. Lumican was localized to the cytoplasm of hepatocytes (arrows), while ASMA was localized to sinusoids.

FIGS. 19A-F are as follows. FIGS. 19A and 19B are photographs of immunohistochemical staining for lumican in two representative liver biopsy tissue samples obtained from patients undergoing transplantation post HCV infection. Lumican is seen localized within hepatocytes and within sinusoids in no particular pattern across the zones. The inset shows that lumican is evenly distributed in normal human liver tissue. FIGS. 19C and 19D are photographs of immunohistochemical staining for lumican in mice undergoing carbon tetrachloride induced chronic liver injury. Lumican expression is upregulated in those hepatocytes clustered around the areas of inflammation and scar tissue. The inset shows that lumican is evenly distributed in normal mouse liver tissue. FIGS. 19E and 19F are graphs plotting lumican gene expression. Lumican gene expression is upregulated five-fold in liver of HCV patients (p<0.001) (FIG. 19E) and seven-fold upregulated in mice undergoing chronic liver injury induced by carbon tetrachloride (p<0.05) (FIG. 19F).

FIG. 20 contains a graph and photograph comparing lumican gene expression in three cells lines, HuH7 (human hepatoma), LX2 (human hepatic stellate), and the H69 (human cholangiocytes), and in normal human liver. Greater expression is seen in hepatocyte cell line as compared to the stellate cells or the cholangiocytes (p<0.05).

FIG. 21 contains a graph and photograph showing that lumican is upregulated in HuH7 cells incubated for 6 hours with 400 μM palmitic (three fold) or stearic acid (2 fold).

FIG. 22 contains graphs and photographs of results from HuH7 or LX 2 cells incubated with or without TGFβ1 at 2 ng/mL over a period of 72 hours. LX2 cells continued to proliferate regardless of exposure to TGFβ1, whereas Huh7 cells exposed to TGFβ1 underwent apoptosis with maximum cell death occurring at 72 hours. Gene expression analysis revealed a 27 fold increase in lumican expression by 72 hours in LX2 cells (top left) as compared to increases of four fold and 14 fold by 48 and 72 hours for HuH7 cells (top right). Western blot analysis of cell lysate and supernatant indicated the presence of two isoforms of different molecular weights at 50 kD and 110 kD that were present predominantly in the supernatant of both the LX2 and the HuH7 cells (bottom left and right). In cell lysate, the 110 kD predominated in the LX2 cells.

FIG. 23 contains photographs of HuH7 or LX2 cells cultured in chamber slides with or without TGFβ1 at 2 ng/mL. Cells were snap frozen at 0, 24, 48, and 72 hours and immunostained for the presence of lumican. Images from the 0 hour and 72 hour time points are presented. Apoptotic fragmented nuclei (white arrows) are visible at 72 hours in HuH7 cells when exposed to TGFβ1, where lumican is seen upregulated. LX2 cells proliferate and stain evenly for lumican. Magnification was either 200× or 100×.

FIG. 24 contains graphs plotting the level of lumican and collagen expression by primary human hepatocytes with or without exposure to TGFβ1.

FIG. 25 contains graphs plotting results the expression levels of collagen 1a1 and beta 1 integrin by HuH7 or LX2 cells cultured with or without TGFβ1 at 2 ng/mL. Cells were harvested at 0, 24, 48, and 72 hours, and expression of collagen 1a1 or beta 1 integrin was measured. Collagen 1 alpha 1 gene expression is upregulated in both LX2 and HuH7 cells. Beta 1 integrin is upregulated in LX2 cells, but remains relatively unchanged in HuH7 cells when exposed to TGFβ1.

FIGS. 26A and 26B are photographs of phase contrast images of HuH7 cells cultured with (FIG. 26A) or without (FIG. 26B) TGFβ1 at 72 hours. No major differences in morphological characteristics were observed. FIGS. 26C and 26D are graphs plotting expression levels of alpha smooth muscle actin (FIG. 26C) and albumin (FIG. 26D) measured at 0, 24, 48, and 72 hours post culture. Alpha smooth muscle actin expression was upregulated four fold, while albumin production decreased four fold by 48 hours when cultured with TGFβ1.

FIG. 27 contains photographs and a graph of results from a comparison of lumican expression in liver tissue from transplant patients with Hepatitis C virus (HCV) and normal liver tissue. The photographs show staining of lumican protein in HCV transplant tissue and normal liver tissue. Lumican gene expression also was examined in liver tissue from transplant patients with HCV and normal liver tissue.

### DETAILED DESCRIPTION

This document provides methods and materials for treating diseases or disorders that are caused by or associated with lumican deposition (e.g., liver fibrosis). For example, this document provides methods and materials for reducing liver fibrosis by reducing lumican expression or activity within a mammal (e.g., a human). This document also provides methods and materials for determining whether or not a test compound reduces lumican polypeptide expression or activity. In addition, this document provides methods and materials for assessing a profibrotic state in a mammal (e.g., a human) with a liver disorder, based on (or based at least in part on) the presence or absence of lumican polypeptide deposition in the mammal

Lumican is a keratan sulfate proteoglycan and belongs to the small leucine-rich proteoglycan (SLRP) family. Lumican is the major keratan sulfate proteoglycan of the cornea, but is also distributed in interstitial collagenous matrices throughout the body. The nucleic acid sequence encoding human lumican is set forth in GenBank GI No. 61742794, and the amino acid sequence of human lumican is set forth in GenBank GI No. 4505047.

As described herein, an inhibitor of lumican can be used to treat a mammal having a disease or disorder that is caused by or associated with lumican deposition (e.g., liver fibrosis). The mammal can be any type of mammal including, without limitation, a mouse, rat, dog, cat, horse, sheep, goat, cow, pig, monkey, or human. An inhibitor of lumican can be any agent that reduces lumican expression (e.g., an siRNA molecule, antisense oligonucleotide, or peptide nucleic acid) or lumican activity (e.g., an inhibitory anti-lumican antibody or matrix metalloproteinase agonists such as prostaglandins).

As described herein, a nucleic acid molecule can be used as an inhibitor of lumican to reduce the expression of a lumican polypeptide. For example, antisense oligonucleotides, siRNA molecules, aptamers, ribozymes, peptide nucleic acid molecules, triplex forming molecules, RNA interference (RNAi) molecules, external guide sequences, and other nucleic acid constructs encoding transcription or translation products can be used to reduce the expression of a lumican polypeptide.

The term “nucleic acid” as used herein encompasses both RNA and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

An “isolated nucleic acid” refers to a nucleic acid that is separated from other nucleic acid molecules that are present in a naturally occurring genome, including nucleic acids that normally flank one or both sides of the nucleic acid in a naturally occurring genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

A nucleic acid construct can comprise a vector containing a nucleotide sequence encoding a transcription or translation product targeting the expression of a lumican polypeptide with any desired transcriptional and/or translational regulatory sequences, such as promoters, UTRs, and 3′ end termination sequences. For example, a polyadenylation region at the 3′-end of the coding region can be included for expression of a polypeptide. In some cases, the polyadenylation region can be derived from a natural gene. Vectors can also include origins of replication, scaffold attachment regions (SARs), markers, homologous sequences, and introns, for example. The vector may also comprise a marker gene that confers a selectable phenotype on cells. The marker may encode antibiotic resistance, such as resistance to kanamycin, G418, bleomycin, or hygromycin.

In some cases, an siRNA molecule, an antisense nucleic acid, or an interfering RNA for reducing the expression of a lumican polypeptide can be similar or identical to at least a part of a lumican allele in a mammal Antisense nucleic acids or interfering RNAs can be about 10 nucleotides to about 2,500 nucleotides in length. For example, nucleic acids described herein can be used as an antisense nucleic acid to a lumican allele. In some cases, the transcription product of a nucleic acid described herein can be similar or identical to the sense coding sequence of a lumican allele, but is an RNA that is unpolyadenylated, lacks a 5′ cap structure, or contains an unsplicable intron.

In some cases, a nucleic acid can have catalytic activity such as a DNA enzyme. For example, a 10-23 DNAzyme can have a cation-dependent catalytic core of 15 deoxyribonucleotides that bind to and cleave target RNA (e.g., a lumican RNA) between an unpaired purine and paired pyrimidine through a de-esterification reaction. The catalytic core can be flanked by complementary binding arms of 6 to 12 nucleotides in length that confer specificity to a lumican mRNA molecule.

In some cases, a nucleic acid can be transcribed into a ribozyme that affects expression of a lumican mRNA. Heterologous nucleic acids can encode ribozymes designed to cleave lumican mRNA transcripts, thereby preventing expression of a lumican polypeptide. Various ribozymes can cleave mRNA at site-specific recognition sequences. For example, hammerhead ribozymes with flanking regions that form complementary base pairs with a lumican mRNA can be used to reduce expression of a lumican polypeptide by cleaving lumican mRNAs at locations containing a 5′-UG-3′ nucleotide sequence.

A nucleic acid described herein can be transcribed into an RNA that is capable of inducing an RNA interference response. In some cases, an interfering RNA can anneal to itself to form, for example, a double stranded RNA having a stem-loop structure. One strand of the stem portion of a double stranded RNA can comprise a sequence that is similar or identical to the sense coding sequence of a lumican polypeptide and that is about 10 nucleotides to about 2,500 nucleotides in length. In some cases, the length of the nucleic acid sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA can comprise an antisense sequence of a lumican polypeptide and can have a length that is shorter, the same as, or longer than the length of the corresponding sense sequence. The loop portion of a double stranded RNA can be from 10 nucleotides to 500 nucleotides in length, for example from 15 nucleotides to 100 nucleotides, from 20 nucleotides to 300 nucleotides or from 25 nucleotides to 400 nucleotides in length. In some cases, the loop portion of the RNA can include an intron.

A nucleic acid can be adapted to facilitate efficient entry into cells. For example, a nucleic acid can be conjugated to and/or complexed with a delivery reagent (e.g., cationic liposomes). In some cases, a nucleic acid can be complexed or conjugated to a protein to confer increased cellular uptake and increased nuclease resistance of oligonucleotides (e.g., Atelocollagen).

As described herein, an antibody can be used as an inhibitor of lumican to reduce the activity of a lumican polypeptide. An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-lumican antibody can bind to a lumican polypeptide at an affinity of at least 10⁴ mol⁻¹ (e.g., at least 10⁵, 10⁶, 10⁷, 10⁸, 10⁹, 10¹⁰, 10¹¹, or 10¹² mol⁻¹).

An anti-lumican antibody provided herein can be prepared using any appropriate method. For example, any substantially pure lumican polypeptide, or fragment thereof (e.g., a truncated lumican polypeptide), can be used as an immunogen to elicit an immune response in an animal such that specific antibodies are produced. Thus, a human lumican polypeptide or a fragment thereof can be used as an immunizing antigen. In addition, the immunogen used to immunize an animal can be chemically synthesized or derived from translated cDNA. Further, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art. See, e.g., Green et al., Production of Polyclonal Antisera, in IMMUNOCHEMICAL PROTOCOLS (Manson, ed.), pages 1-5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan et al., Unit 9, CURRENT PROTOCOLS IN

IMMUNOLOGY, Wiley Interscience, 1994).

The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 2.9.3; Barnes et al., Purification of Immunoglobulin G (IgG), in METHODS IN MOLECULAR BIOLOGY, Vol. 10, pages 79-104 (Humana Press 1992).

Once hybridoma clones that produce antibodies to an antigen of interest (e.g., a lumican polypeptide) have been selected, further selection can be performed for clones that produce antibodies having a particular specificity. For example, clones can be selected that produce antibodies that preferentially bind to a lumican polypeptide and inhibit lumican polypeptide activity (e.g., the ability to support the formation of liver fibrosis).

The antibodies provided herein can be substantially pure. The term “substantially pure” as used herein with reference to an antibody means the antibody is substantially free of other polypeptides, lipids, carbohydrates, and nucleic acid with which it is naturally associated in nature. Thus, a substantially pure antibody is any antibody that is removed from its natural environment and is at least 60 percent pure. A substantially pure antibody can be at least about 65, 70, 75, 80, 85, 90, 95, or 99 percent pure.

This document also provides methods and materials related to treating mammals (e.g., humans) likely to develop a disease or disorder that is caused by or associated with lumican deposition (e.g., liver fibrosis). A mammal can be identified as having or being likely to develop such as disease or disorder using standard clinical techniques. For example, analysis by liver biopsy, radiographic determination of liver stiffness (e.g., by ultrasound or magnetic resonance elastography) or by blood parameter algorithms (e.g., FibroTest) can be used to determine whether or not a human is likely to develop liver fibrosis. As described herein, a mammal identified as having or being susceptible to developing a disease or disorder that is caused by or associated with lumican deposition (e.g., liver fibrosis) can be treated by administering an inhibitor of lumican.

Agents that can inhibit lumican expression or activity in cells can be identified by screening candidate agents (e.g., from synthetic compound libraries and/or natural product libraries). Candidate agents can be obtained from any commercial source and can be chemically synthesized using methods that are known to those of skill in the art. Examples of candidate agents include, without limitation, polypeptides, peptidomimetics, peptoids, small inorganic molecules, small non-nucleic acid organic molecules, nucleic acid molecules such as antisense nucleic acids, siRNAs, ribozymes, or triple helix molecules, or other drugs. Candidate agents can be screened and characterized using in vitro cell-based assays and/or in vivo animal models. For example, a candidate agent can be assessed for the ability to reduce lumican polypeptide expression using standard assays such as Western Blots, ELISAs, or immunohistochemistry. In some cases, lumican expression can be measured by mRNA analysis (e.g., RT-PCR). In some cases, collagen fibril assembly, fibrillar collagen growth, and/or collagen abundance can be assessed to determine whether or not a candidate agent reduces lumican polypeptide expression or activity.

An inhibitor of lumican can be administered to a mammal alone or in combination with other agents such as another inhibitor of lumican. For example, a composition containing an anti-lumican antibody can be administered to a mammal in need of treatment for a liver condition. Such a composition can contain additional ingredients including, without limitation, pharmaceutically acceptable vehicles. A pharmaceutically acceptable vehicle can be, for example, saline, water, lactic acid, or mannitol.

A composition containing an inhibitor of lumican can be administered to mammals by any appropriate route, such as enterally (e.g., orally), parenterally (e.g., subcutaneously, intravenously, intradermally, intramuscularly, or intraperitoneally), intracerebrally (e.g., intraventricularly, intrathecally, or intracisternally) or intranasally (e.g., by intranasal inhalation).

Suitable formulations for oral administration can include tablets or capsules prepared by conventional means with pharmaceutically acceptable excipients such as binding agents (e.g., pregelatinized maize starch, polyvinylpyrrolidone, or hydroxypropyl methylcellulose), fillers (e.g., lactose, microcrystalline cellulose, or calcium hydrogen phosphate), lubricants (e.g., magnesium stearate, talc, or silica), disintegrants (e.g., potato starch or sodium starch glycolate), or wetting agents (e.g., sodium lauryl sulfate). Tablets can be coated by methods known in the art. Preparations for oral administration also can be formulated to give controlled release of the agent.

A composition containing an inhibitor of lumican can be administered to a mammal in any amount, at any frequency, and for any duration effective to achieve a desired outcome (e.g., to reduce lumican expression, lumican activity, or liver fibrosis). In some cases, a composition containing an inhibitor of lumican can be administered to a mammal to reduce liver fibrosis in the mammal by 5, 10, 15, 20, 25, 30, 35, 40, 45, 50, 55, 60, 65, 70 percent or more. An effective amount of an inhibitor of lumican can be any amount that reduces lumican expression, lumican activity, and/or liver fibrosis without producing significant toxicity to a mammal Typically, an effective amount of an inhibitor of lumican can be any amount greater than or equal to about 10 μg provided that that amount does not induce significant toxicity to the mammal upon administration. In some cases, an effective amount of an inhibitor of lumican can be between 1 μg and 500 mg (e.g., between 1 μg and 250 mg, between 1 μg and 200 mg, between 1 μg and 150 mg, between 1 μg and 100 mg, between 1 μg and 50 mg, between 1 μg and 10 mg, between 1 μg and 1 mg, between 1 μg and 100 μg, between 1 μg and 50 μg, between 5 μg and 100 mg, between 10 μg and 100 mg, between 100 μg and 100 mg, or between 10 μg and 10 mg). Various factors can influence the actual effective amount used for a particular application. For example, the frequency of administration, duration of treatment, use of multiple treatment agents, route of administration, and severity of the liver condition may require an increase or decrease in the actual effective amount administered.

The frequency of administration of an inhibitor of lumican can be any frequency that reduces lumican expression, lumican activity, and/or liver fibrosis without producing significant toxicity to the mammal For example, the frequency of administration can be from about three times a day to about twice a month, or from about once a week to about once a month, or from about once every other day to about once a week, or from about once a month to twice a year, or from about four times a year to once every five years, or from about once a year to once in a lifetime. The frequency of administration can remain constant or can be variable during the duration of treatment. For example, an inhibitor of lumican can be administered daily, twice a day, five days a week, or three days a week. An inhibitor of lumican can be administered for five days, 10 days, three weeks, four weeks, eight weeks, 48 weeks, one year, 18 months, two years, three years, or five years. A course of treatment can include rest periods. For example, an inhibitor of lumican can be administered for five days followed by a ten-day rest period, and such a regimen can be repeated multiple times. As with the effective amount, various factors can influence the actual frequency of administration used for a particular application. For example, the effective amount, duration of treatment, use of multiple treatment agents, route of administration, and severity of the liver condition may require an increase or decrease in administration frequency.

An effective duration for administering an inhibitor of lumican can be any duration that reduces lumican expression, lumican activity, and/or liver fibrosis without producing significant toxicity to the mammal Thus, the effective duration can vary from several days to several weeks, months, or years. In general, the effective duration for the treatment of a liver condition can range in duration from several days to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the liver condition.

This document also provides methods and materials for identifying agents that can be used to treat a mammal having or being likely to develop a liver condition that is caused by or associated with lumican deposition (e.g., liver fibrosis). For example, a lumican polypeptide expression assay can be used to identify agents that can be used to treat a mammal having or being likely to develop a liver condition that is caused by or associated with lumican deposition (e.g., liver fibrosis). In addition, an animal model resistant to liver fibrosis (e.g., a lumican knockout mice) can be used as a control for confirming an agent's ability to treat a liver condition that is caused by or associated with lumican deposition.

The invention will be further described in the following examples, which do not limit the scope of the invention described in the claims.

### EXAMPLES

### Example 1

### Lumican Expression is Upregulated in Multiple Disease Etiologies

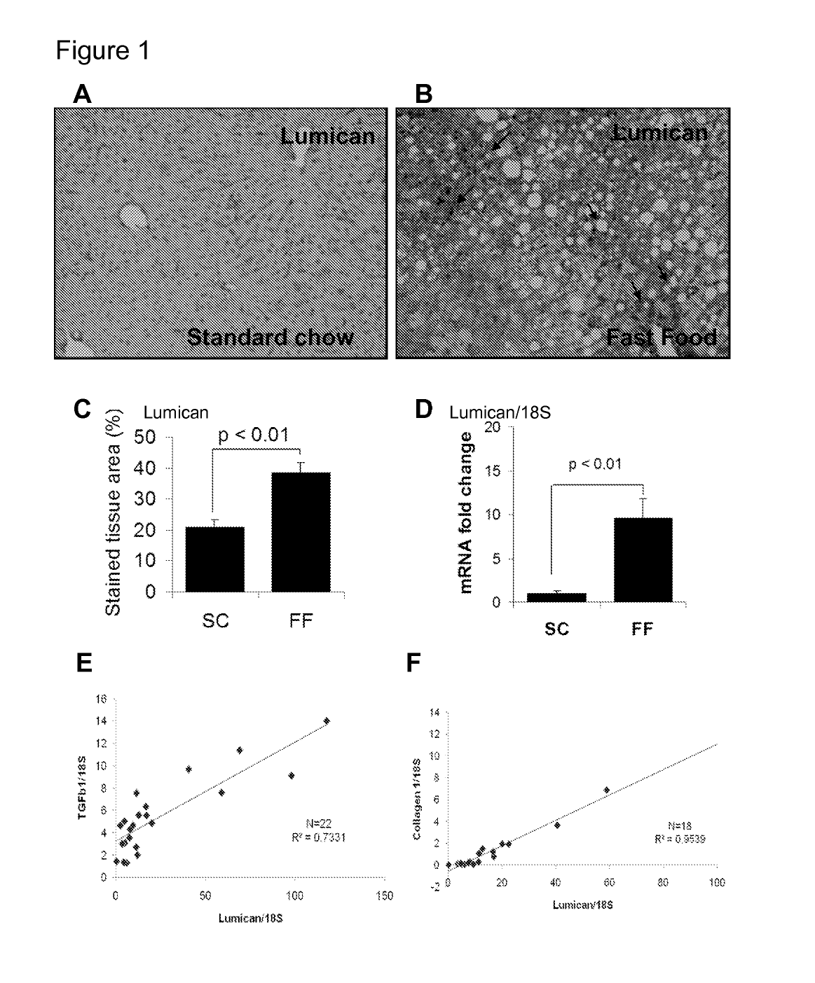

Lumican regulation in response to liver injury was examined in two rodent models of differing severity: (1) diet induced obesity leading to steatohepatitis and (2) acute necro-inflammation induced by hepatotoxin CC14. In the first instance, prolonged feeding with the FF diet was shown to induce steatohepatitis and fibrosis. Immunohistochemical staining for lumican (FIG. 1B) in those animals fed the ‘fast food’ (FF) diet showed a mosaic pattern very similar to that seen in NASH patients with sinusoidal and cytoplasmic staining of hepatocytes. Staining was intense in areas around fat vacuoles (five o'clock arrows) and around areas of inflammation (seven o'clock arrows). In animals fed the SC diet (FIG. 1A), however, lumican was localized specifically to hepatic sinusoids. Digital image analysis of immunostained sections (FIG. 1C) and gene expression (FIG. 1D) indicated a significantly higher lumican expression in FF fed mice as compared to those animals fed the SC (p<0.01). Additionally, in this group of animals, lumican gene expression correlated positively with gene expression of TGFβ1 (r²=0.73) and collagen (r²=0.95) (FIGS. 1E and 1F), suggesting a possible association with pro-fibrotic signaling pathways.

### Example 2

### Lumican Deficiency Protects Against Hepatic Fibrosis

Transgenic mouse strains for lumican knockout mouse (Lumican −/−), lumican heterozygous mouse (Lumican +/−) and wild type lumican mouse (Lumican +/+) were obtained to test whether lumican deficiency protects against hepatic fibrosis (Chakravarti et al., J. Cell Biol., 141(5):1277-86 (1998)).

Mice were injected twice weekly intraperitoneally with 1 mg/kg of carbon tetrachloride (CC14) and control mice were injected with vehicle (corn oil) only. Time points for analysis were scheduled at 1 week, 1 month and 3 months. Mice were sacrificed 2 days after the last CC14 injection.

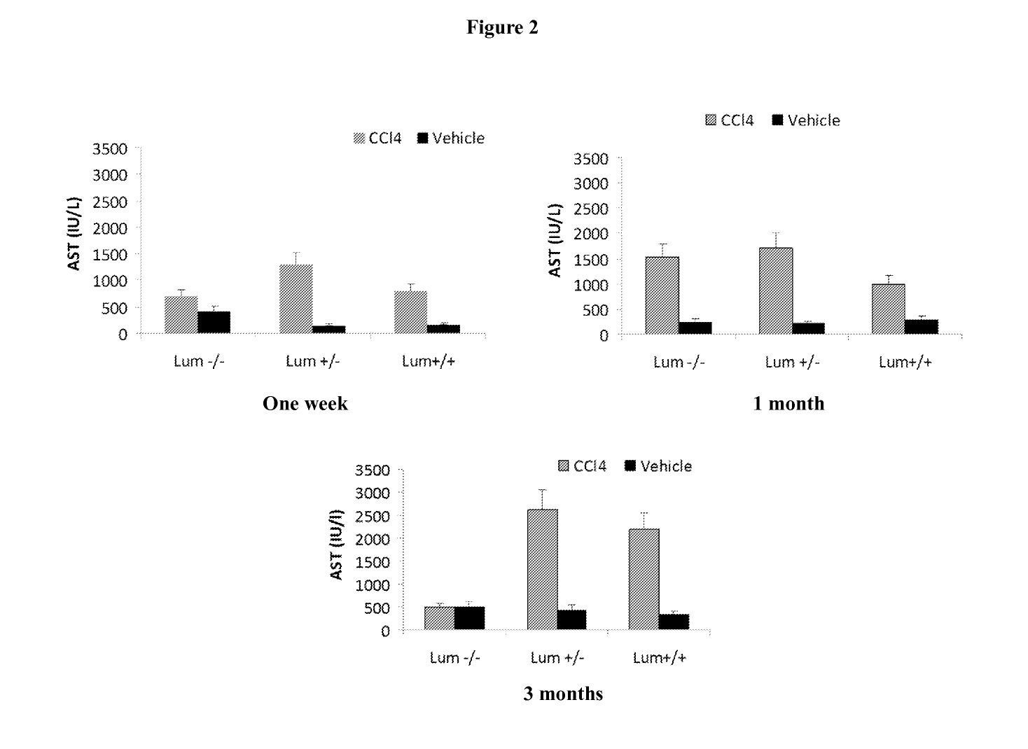

Serum aspartate aminotransferase (AST) was measured by kinetic ultraviolet method (FIG. 2). Results show that serum AST levels in Lumican −/− mice were decreased at 3 months when compared to levels in Lumican +/− and Lumican +/+. Low levels of AST are normally found in the blood. When body tissue or an organ such as the heart or liver is diseased or damaged, additional AST is released into the bloodstream. The amount of AST in the blood is directly related to the extent of the tissue damage. These results indicate that lumican inhibition may have an anitinflammatory effect in the liver.

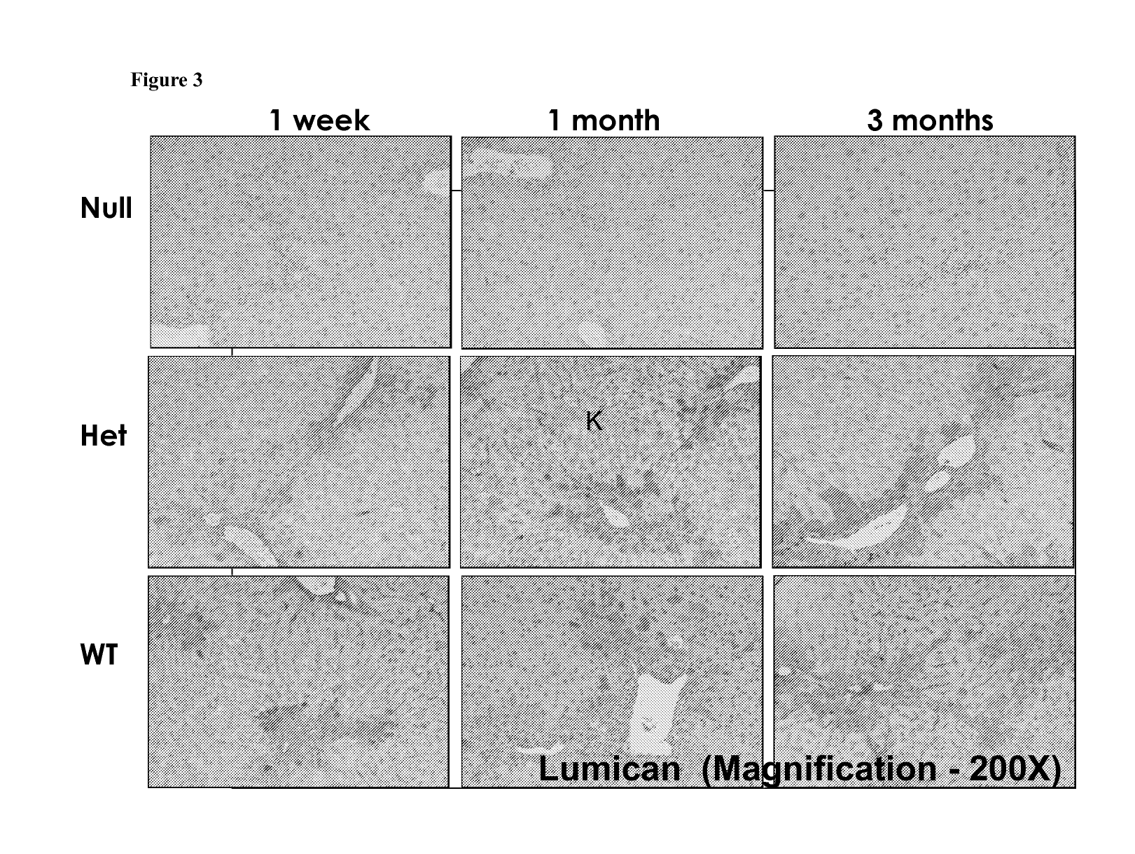

Immunohistochemistry with an anti-lumican antibody (R&D systems, Minneapolis, Minn., USA) on liver tissues of mice show that lumican expression is increased at all time points in Lumican +/− and Lumican +/+ when compared with Lumican −/− mice (FIG. 3).

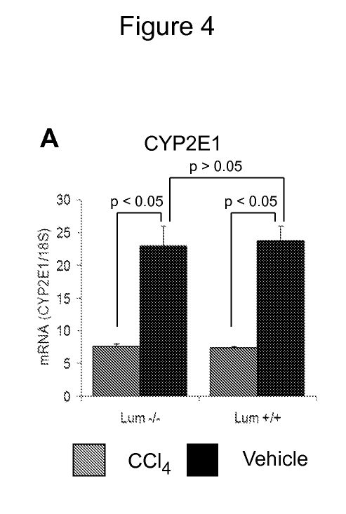

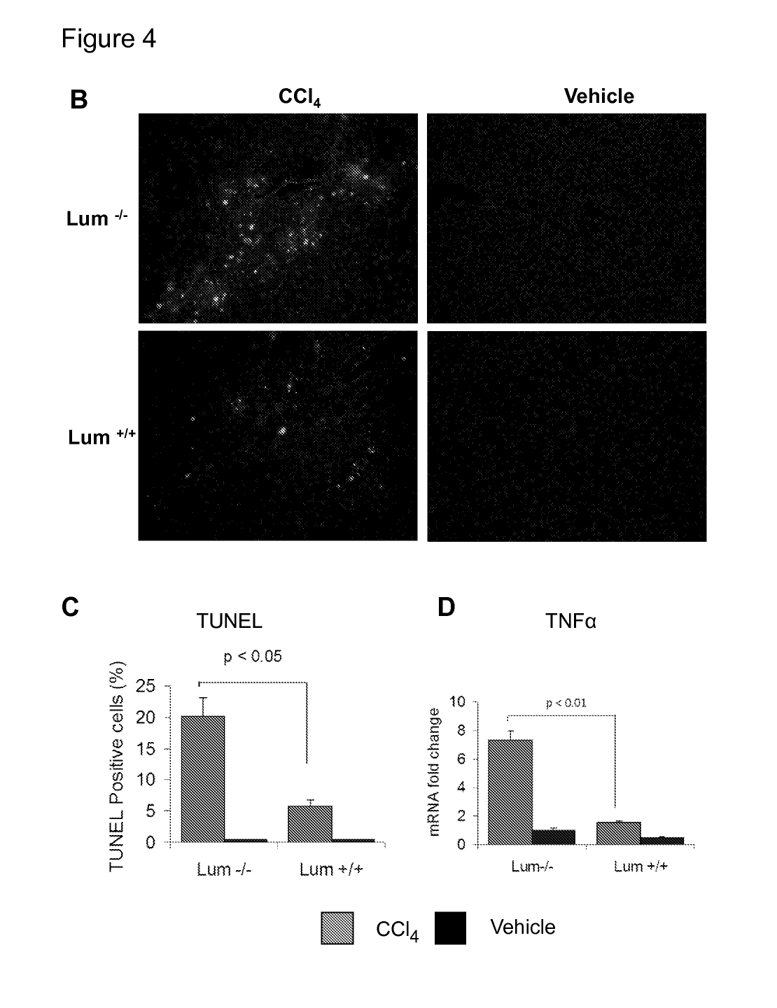

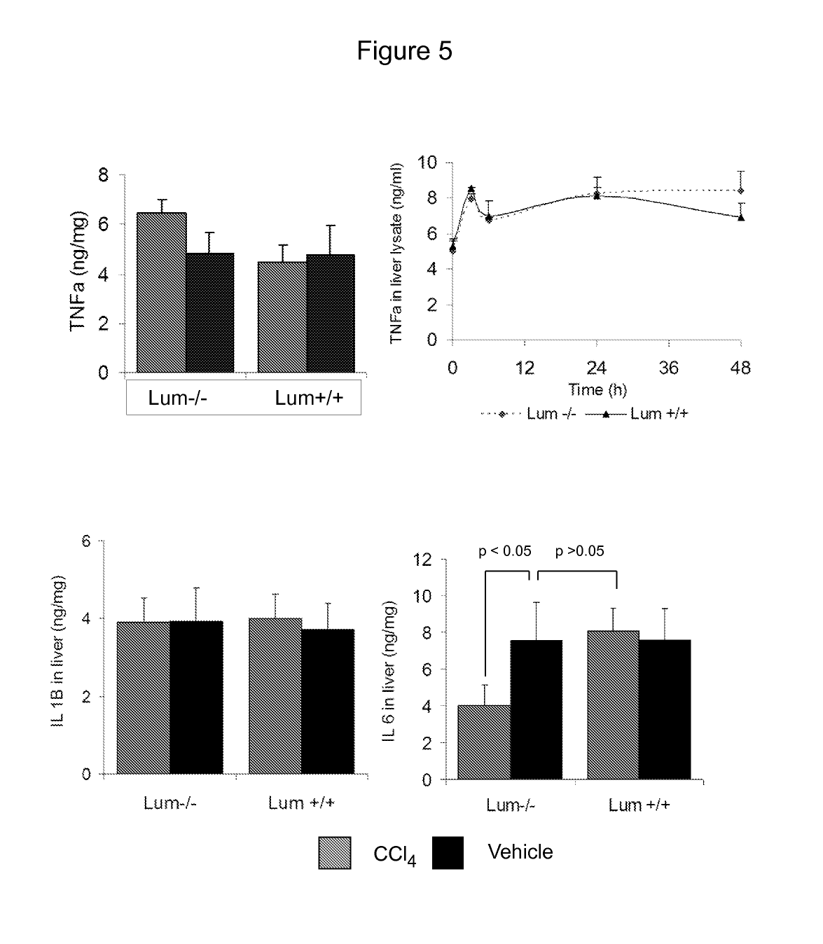

Since hepatotoxicity of CC14 is dependent on its conversion to CC13, expression of CYP2E1, the cytochrome involved in biotransformation of CC14 to CC13, was confirmed to be similarly regulated in both genotypes (FIG. 4A). Apoptosis measured by immunohistochemical staining for TUNEL in formalin preserved paraffin embedded tissue sections (FIG. 4B) was significantly higher in null mice (p<0.001) as compared to their wild type counterparts (FIG. 4C). Among the inflammatory cytokines, hepatic gene expression of TNFα, a known mediator of apoptosis, was significantly higher in null animals (FIG. 4D). Only TNFα was insignificantly higher (p=0.09) among the panel of inflammatory cytokines examined by ELISA within the liver (FIG. 5).

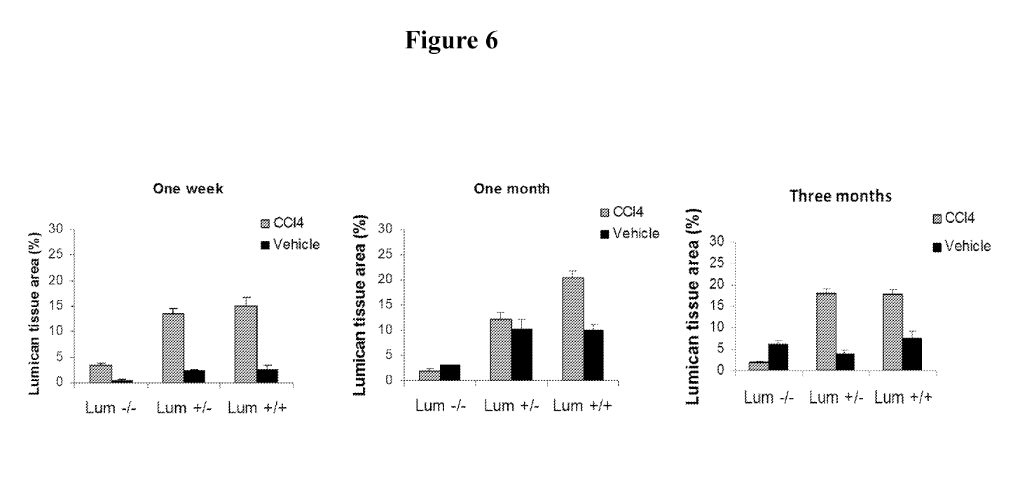

A histological analysis of lumican protein expression in liver, obtained using anti-lumican (R&D systems, Minneapolis, Minn., USA), demonstrates that the percentage of tissue area showing expression of lumican protein significantly increases over time in both the Lumican +/− and Lumican +/+ groups but not in the Lumican −/− group (FIG. 6).

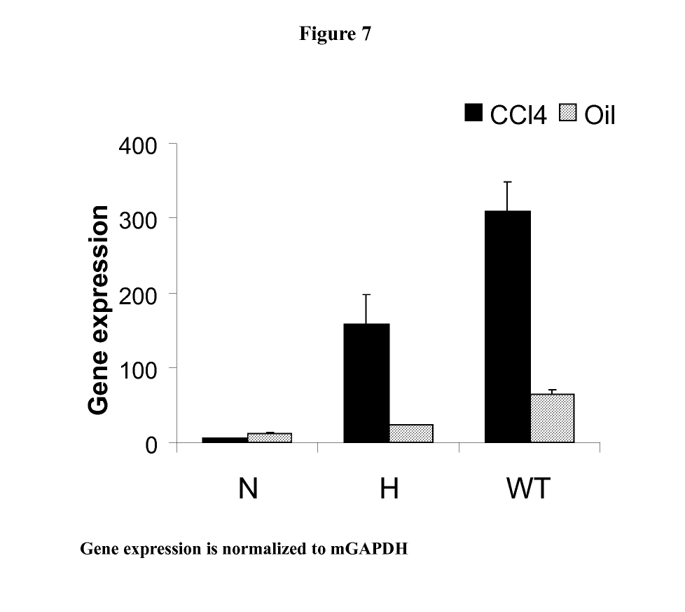

In addition, gene expression of lumican was analyzed across all three groups of mice at the 1 month time point. Gene expression was normalized to mGAPDH. Results demonstrate significantly lower expression in the Lumican −/− group versus Lumican +/− and Lumican +/+ (FIG. 7).

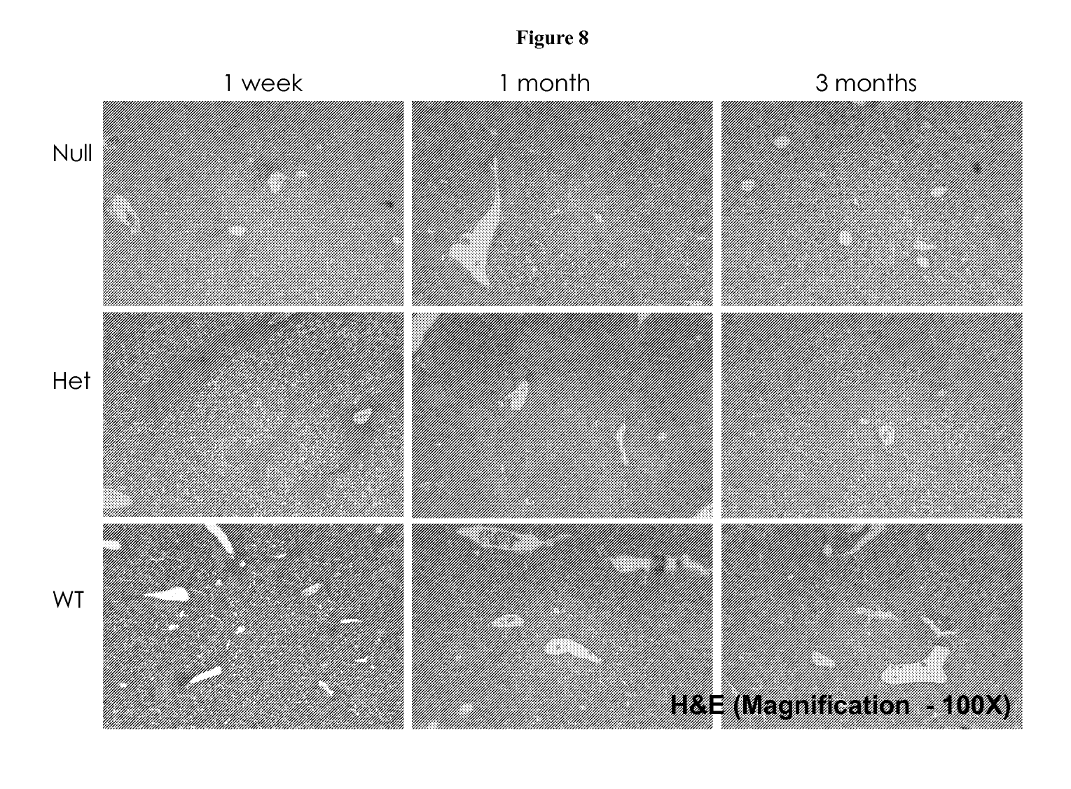

Liver tissue samples were obtained from the different mice groups and examined with various stains. An HE stain was performed on liver tissue from of Lumican −/−, Lumican +/− and Lumican +/+ mice treated with CC14 at different time points (FIG. 8). This figure demonstrates a necroinflammatory response in all animals receiving CC14.

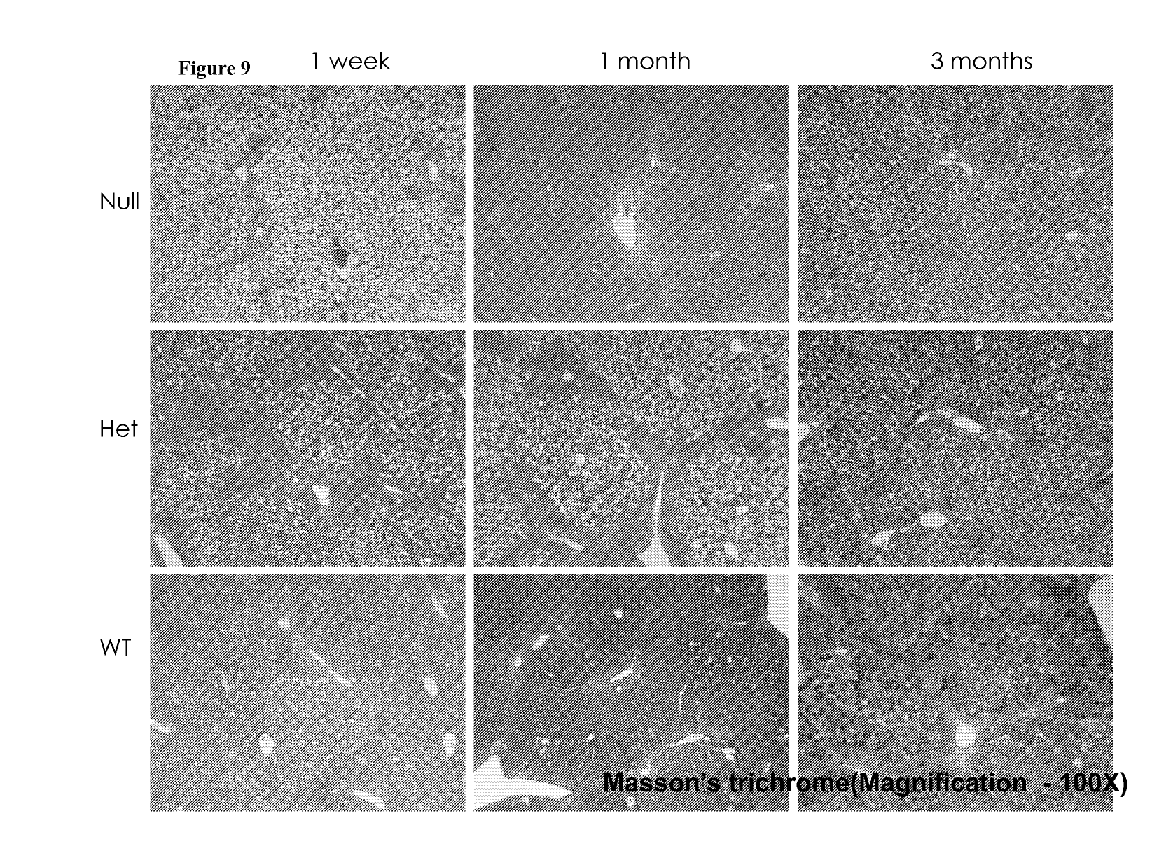

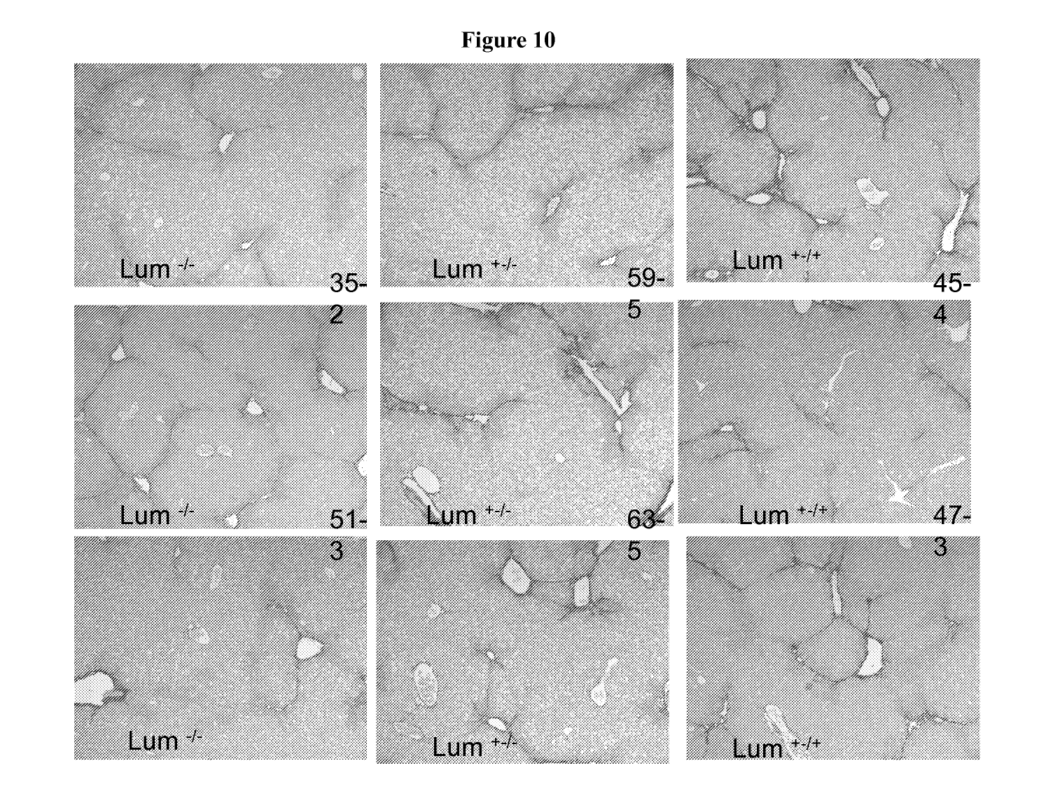

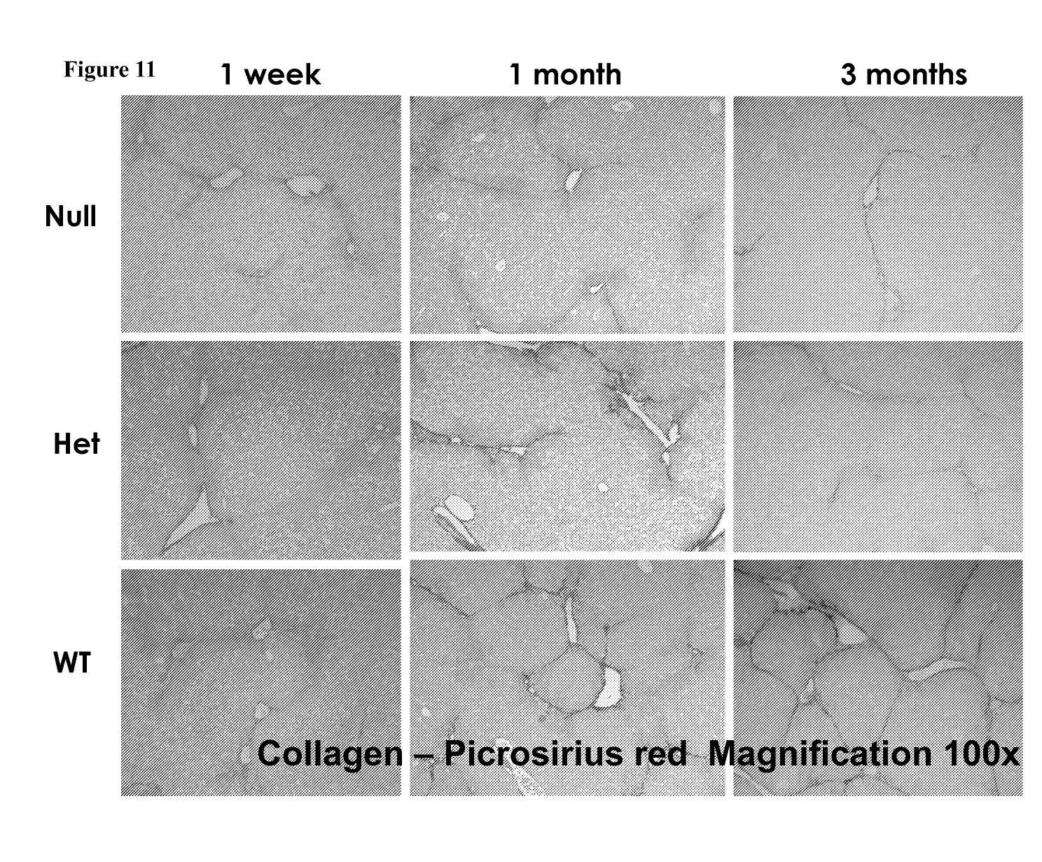

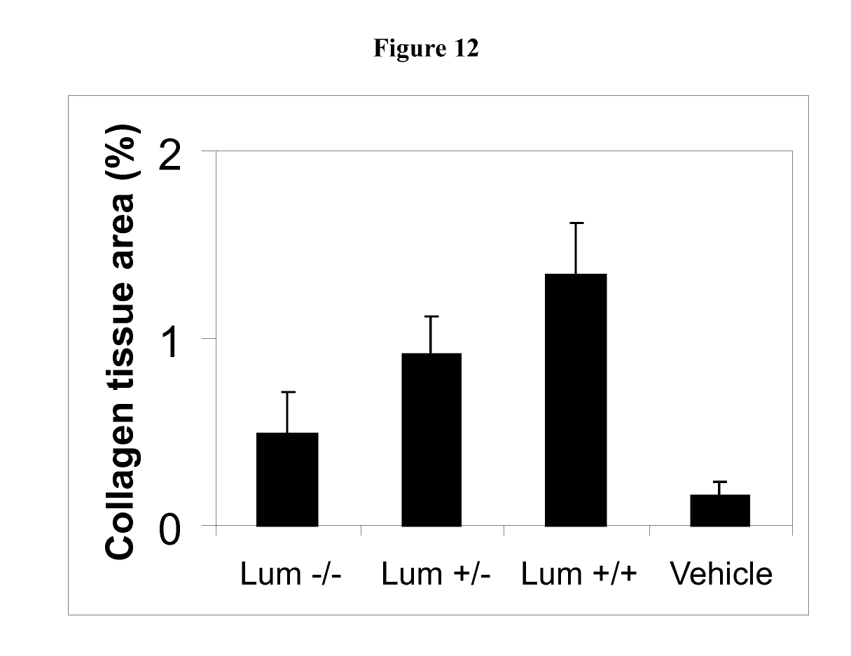

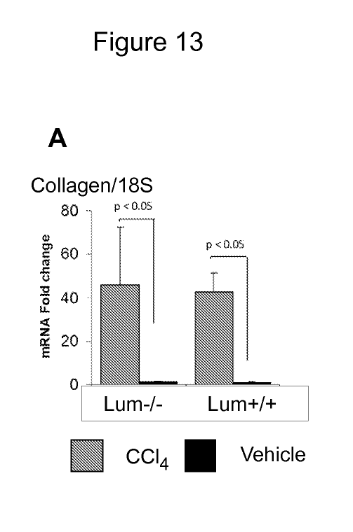

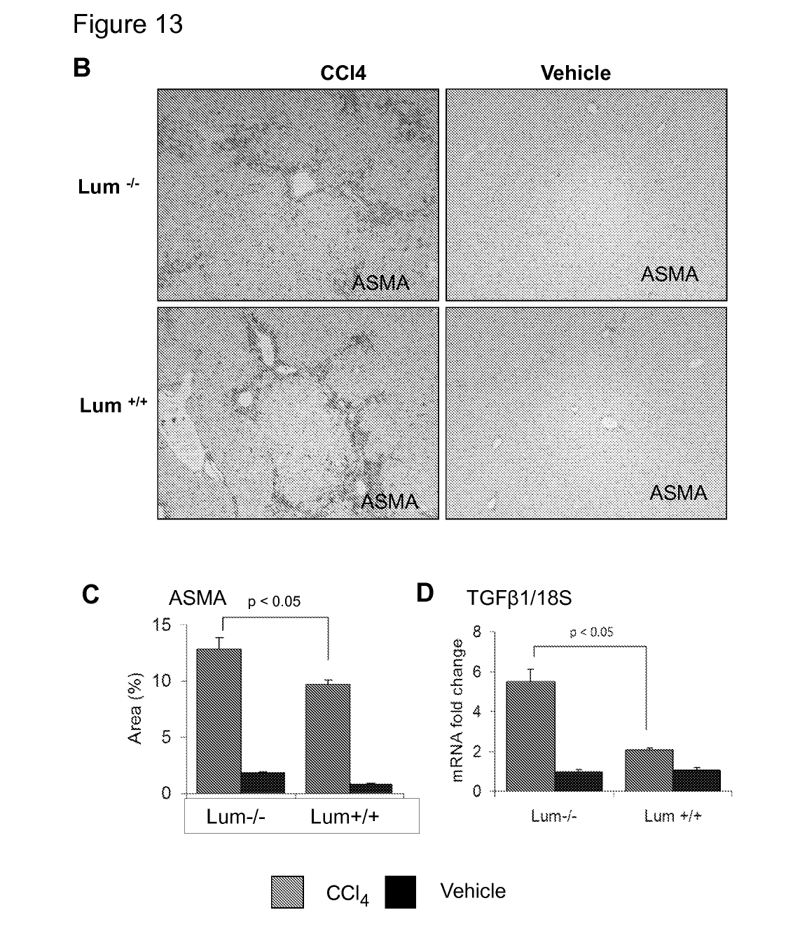

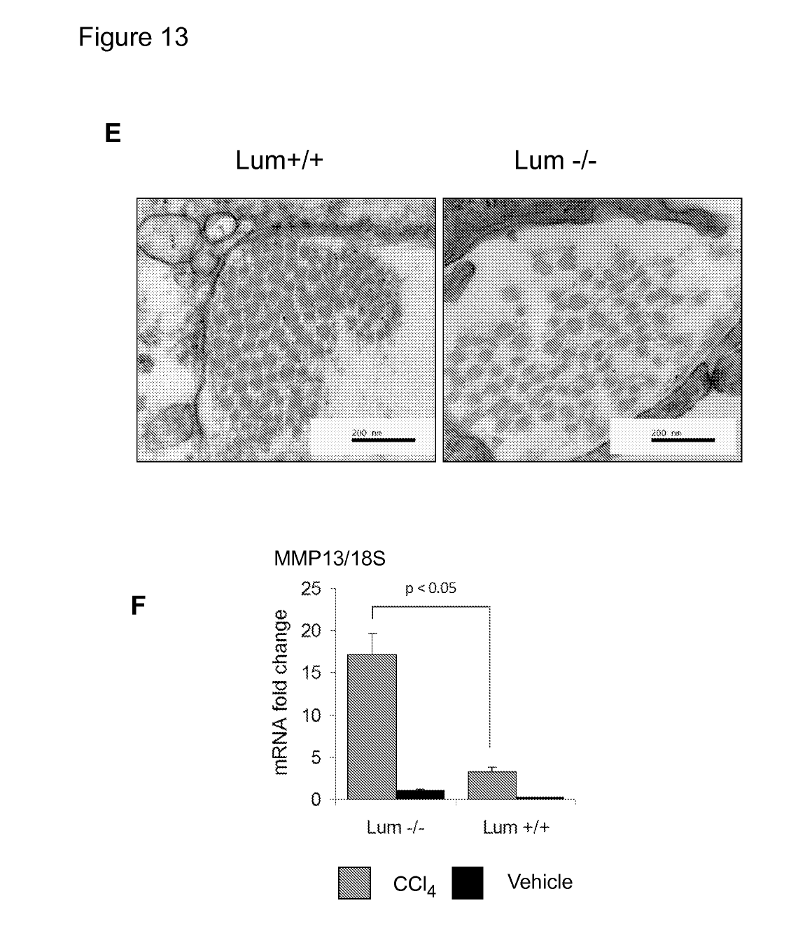

A Masson's trichrome stain (FIG. 9) and a Picrosirius red stain (FIG. 10) were performed on liver tissue from of Lumican −/−, Lumican +/− and Lumican +/+ mice treated with CC14 at different time points. These results demonstrate near complete lack of stainable fibrosis in Lumican −/− animals. Fibrosis is markedly increased in the Lumican +/− and +/+ animals during treatment with CC14 (FIG. 11). A histological analysis of collagen protein expression also shows increased percentage of collagen protein expression in tissue in Lumican +/− and Lumican −/− groups (FIG. 12). Gene expression analysis, however, indicated that collagen expression was upregulated both in null and wild type animals as compared to controls (FIG. 13A).

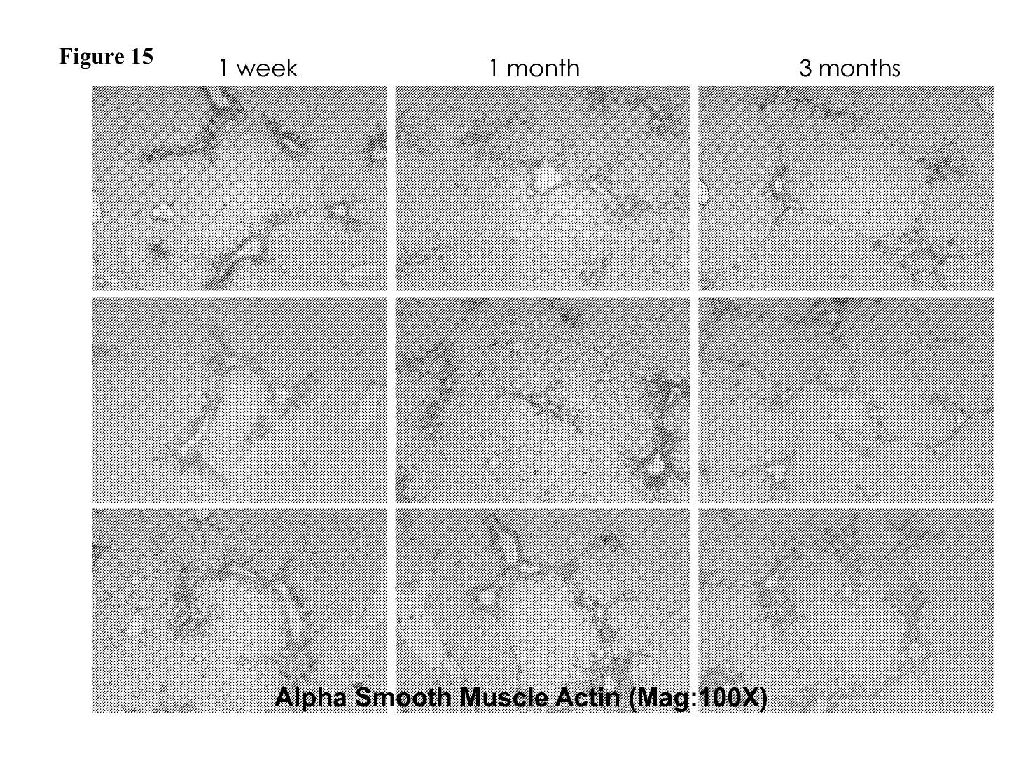

Staining for alpha smooth muscle actin was performed in liver tissue for Lumican −/−, Lumican +/− and Lumican +/+ mice treated with CC14 at different time points (FIG. 15). This stain showed that the effect of lumican in inhibiting hepatic fibrosis is not mediated through inhibition of hepatc stellate cells, which were equally activated in −/−, +/− and +/+ animals.

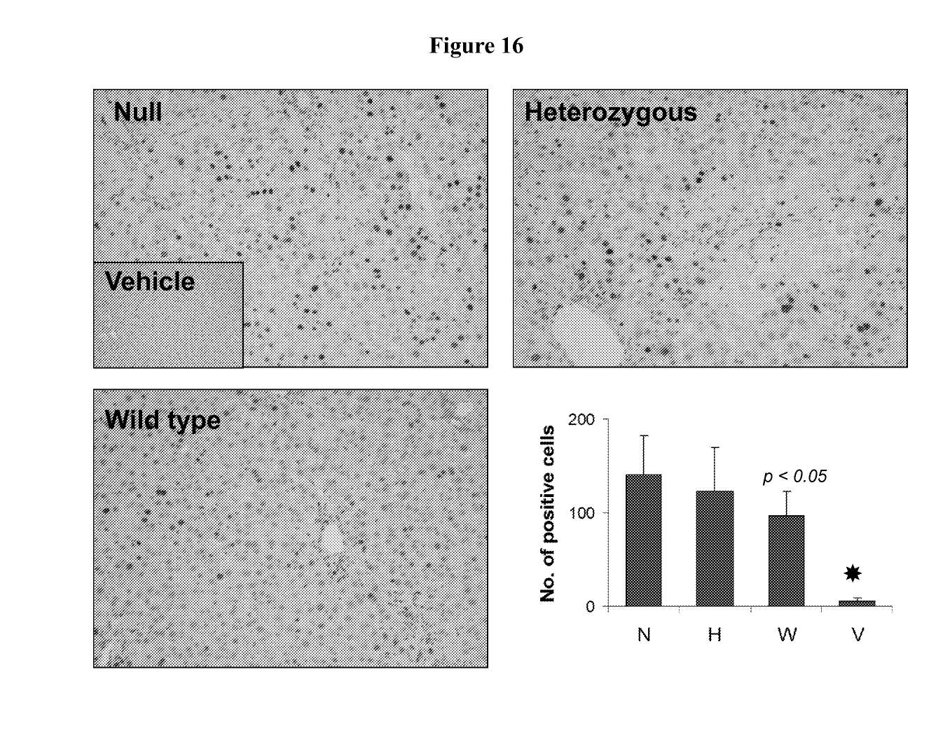

An immunohistochemical stain for Ki67, a marker of cell proliferation, was performed (FIG. 16). This figure demonstrates increased proliferative response of hepatocytes in Lumican −/− and Lumican +/− animals when compared to Lumican +/+ animals.

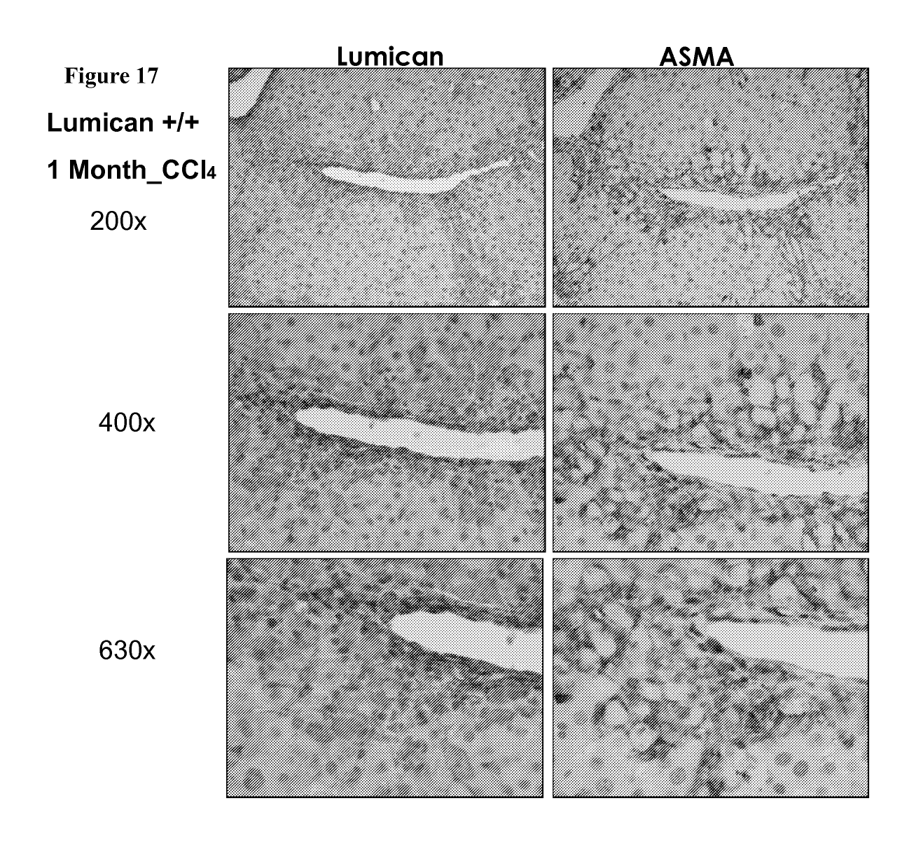

FIG. 17 is an overlay immunohistochemical stain of liver tissue from Lumican +/+ mice treated with CC14. Samples were stained with anti-smooth muscle actin and anti-lumican. ASMA staining is apparent in the same anatomical distribution as lumican staining, corresponding to the portal tracts and zone 3. These results demonstrate that lumican is required for hepatic fibrosis in response to carbon tetrachloride mediated injury. It presents evidence of a dose effect of lumican and fibrotic response to CC14. These results also indicate that lumican is not protective against initial necro-inflammatory response to CC14.

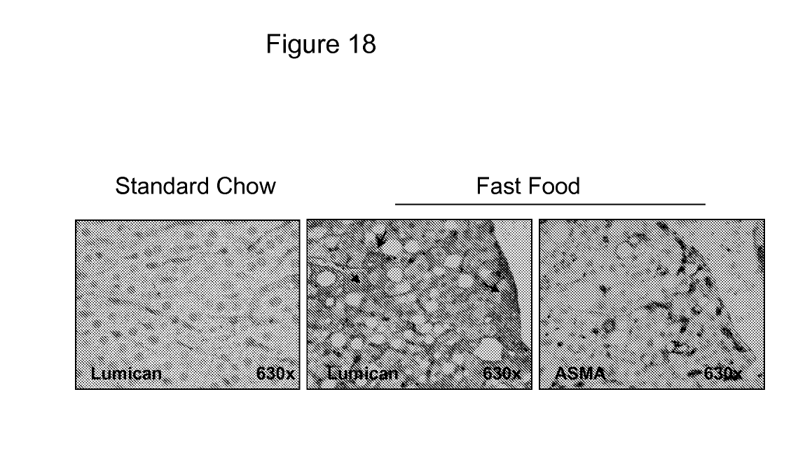

In addition, the serial sections of liver with CC14 mediated liver injury stained both for alpha smooth muscle actin (ASMA), an established marker of activated stellate cells, and for lumican were compared (FIG. 17). Lumican was localized to the hepatic sinusoids in the animals given vehicle alone. However, in animals administered CC14, cytoplasmic staining for lumican within individual hepatocytes around areas of inflammation (FIG. 17) was distinct from the sinusoidal staining of ASMA suggesting that when injured, hepatocytes are the primary source of lumican in the liver. Similar observations were recorded for animals on the fast food and standard chow diets (FIG. 18).

Although injured hepatocytes are known to secrete collagen, the primary source of collagen within the liver are activated hepatic stellate cells. The samples were examined for evidence of hepatic stellate cell activation by staining liver tissue sections for ASMA (FIG. 13B). ASMA was significantly higher in null animals (FIG. 13C). TGFβ1, the cytokine best known to drive stellate cell activations, was also significantly higher in null animals (FIG. 13D). The matrix is known to undergo constant turnover under the combined influence of matrix metallo-proteases (MMP) and tissue inhibitor of metallo-proteases (TIMP). Among the metallo-proteases, MMP13, the rodent equivalent of human MMP1, was four-fold significantly increased (p<0.05) in lumican null animals (FIG. 13F), suggesting that the secreted collagen was being eliminated from the matrix. MMP9 and TIMP1 were insignificantly higher in null animals.

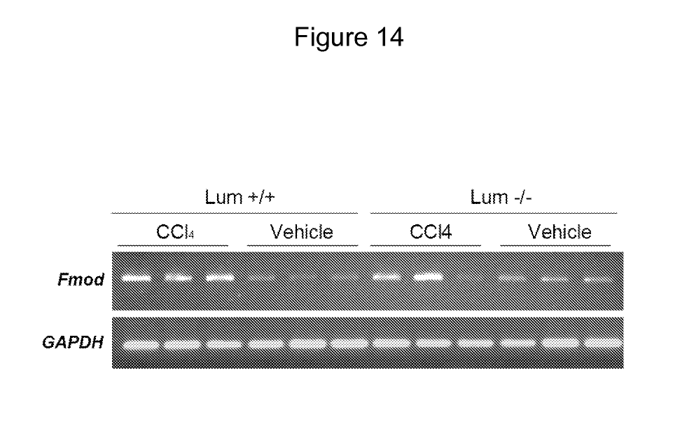

Since SLRP's include other family members such as fibromodulin, which binds to collagen, the possibility of fibromodulin compensating for the lack of lumican in the null animals was examined Fibromodulin was increased in CC14 treated animals of both genotypes as compared to the animals given vehicle alone (FIG. 14). However, it was not over-expressed in null animals as compared to the wild type animals. It is likely that the increased expression of MMP13 in lumican null animals contributed to degradation of both collagen and fibromodulin, thereby inhibiting the accumulation of collagen in the matrix.

Ultra-structural imaging of collagen fibrils in lumican null and wild type animals indicated a marked difference in their structural organization. In null animals, the collagen fibrils appeared scattered, widely dispersed and of different diameters (FIG. 13E). By contrast, in lumican wild type animals, collagen fibrils were evenly spaced and were of uniform diameter. These results demonstrate that collagen fibril structural assembly was impaired in lumican null animals.

### Example 3

### Differential Expression of Lumican in a Hepatocyte Cell line (HuH7), a Stellate Cell Line (LX2), and a Cholangiocyte Cell Line (H69)

As the cellular sources of lumican within the liver and its response to pro-fibrotic and inflammatory stimuli are unknown, lumican expression was examined within three cell lines of differing hepatic origin: hepatocytes (HuH7), cholangiocytes (H69), and hepatic stellate cells (LX2). In addition, the response of lumican expression to pro-fibrotic transforming growth factor β1 (TGFβ1) and pro-inflammatory signaling by saturated free fatty acids (FFA) was measured.

### Hepatic Cell Lines and Culture

The hepatocyte cell line HuH7, the cholangiocyte cell line H69, and the hepatic stellate cell line LX2 were grown to confluence in DMEM with 10% fetal bovine serum and antibiotics Penicillin (100 U/mL) and Streptomycin (10 μg/mL) at 37° C. and 5% CO₂ Cells were harvested, washed in cold phosphate buffered saline and snap frozen. In a second experiment to examine the effect of the cytokine TGFβ1 (R& D systems, MN, USA), HuH7, or LX2 cells were seeded at 0.2 million cells per well in a 6 well plate for 24 hours, and were serum starved for a further 24 hours following which one group received TGFβ1 at 2 ng/mL while a second group that did not receive TGFβ1 served as the control. At 24, 48 and 72 hours, cells were trypsin digested, washed three times with ice cold phosphate buffered saline and quick frozen at −80° C. in two separate lots for either protein or RNA extraction. In separate experiments, palmitic or stearic acid was added to the culture media of HuH7 cells at a concentration of 400 μM (Cazanave et al., J. Biol. Chem., 284:26591-26602 (2009)). All experiments were carried out in triplicate and results are reported as the average of triplicates.

### RNA Isolation and Real-Time PCR

Total RNA was isolated from cells collected from the cell culture experiments (described herein) or from liver tissue samples collected from patient or from mouse experiments (described herein) using the RNeasy Plus kit as per the manufacturer's instructions (Qiagen, GmbH, Germany). 500 ng of total RNA was reverse transcribed into cDNA using random hexamers (Transcriptor High Fidelity cDNA synthesis kit, Roche, USA). Real time PCR was carried on a LightCycler (Roche, CA, USA) using equal quantities of template cDNA in a total volume of 20 μL using Lightcycler FastStart DNA Master SYBR green1 (Roche, Indianapolis, USA). The primers used are presented in Table 1. Expression of 18S rRNA was used as the internal standard (Quantum RNA 18S Internal Standards, Ambion Inc, Austin, Tex., USA). Standard curves were generated for each optimized assay using known PCR copy numbers to produce a linear plot of threshold cycle (Ct) against log dilution. Data for lumican is presented as lumican copy number normalized against the 18S absolute copy number where indicated. For all other gene expression assays, results are presented as fold change as represented by the expression 2^{-ΔΔCt} (Schmittgen & Livak, Nat. Protoc., 3:1101-1108 (2008)).

TABLE 1
Primers.

| | | GenBank ® | |
|---|---|---|---|
| | | Accession | |
| Gene | Species | No. | Primers |
| Lumican | Human | NM_002345.3 | F: |
| | | | CTTCAATCAGATAGCCAGACTGC |
| | | | (1) |
| | | | R: |
| | | | AGCCAGTTCGTTGTGAGATAAAC |
| | | | (2) |
| Lumican | Rodent | NM_008524.2 | F: |
| | | | TCGAGCTTGATCTCTCCTAT |
| | | | (3) |
| | | | R: |
| | | | TGGTCCCAGGATCTTACAGAA |
| | | | (4) |
| 18S | Rodent | NR_003278.1 | F: |
| | | | CTCAACACGGGAAACCTCAC |
| | | | (5) |
| | Human | NR_003286.1 | R: |
| | | | CGCTCCACCAACTAAGAACG |
| | | | (6) |
| COLL1a1 | Human | NM_000088.3 | F: |
| | | | GGTAACAGCGGTGAACCTG |
| | | | (7) |
| | | | R: |
| | | | GAGCTCCTCGCTTTCCTTC |
| | | | (8) |
| Coll1a1 | Mouse | NM_007742.3 | F: |
| | | | CTCCTGGCAAGAATGGAGAT |
| | | | (9) |
| | | | R: |
| | | | AATCCACGAGCACCCTGA |
| | | | (10) |
| Cyp2e1 | Mouse | NM_021282 | F: |
| | | | GGAACACCTTAAGTCACTGGACA |
| | | | (11) |
| | | | R: |
| | | | TGGGTTCTTGGCTGTGTTTT |
| | | | (12) |
| TNFα | Mouse | NM_013693.2 | F: |
| | | | TGCCTATGTCTCAGCCTCTTC |
| | | | (13) |
| | | | R: |
| | | | GAGGCCATTTGGGAACTTCT |
| | | | (14) |
| TGFβ1 | Mouse | NM_011577.1 | F: |
| | | | TGGAGCAACATGTGGAACTC |
| | | | (15) |
| | | | R: |
| | | | CAGCAGCCGGTTACCAAG |
| | | | (16) |
| Mmp13 | Mouse | NM_008607.2 | F: |
| | | | ACCAGTCTCCGAGGAGAAACTAT |
| | | | (17) |
| | | | R: |
| | | | GGACTTTGTCAAAAAGAGCTCAG |
| | | | (18) |
| Fmod | Mouse | NM_021355.3 | F: |
| | | | TGGAGGGCCTGGAGAACCTCAC |
| | | | (19) |
| | | | R: |
| | | | GTGCAGAAGCTGCTGATGGAGAA |
| | | | (20) |

SEQ ID NO is in parenthesis.

### Western Blot Analysis

Total protein from snap frozen cell pellets were extracted in lysis buffer containing 30 mM Tris-HCl (pH7.4), 150 mM sodium chloride, 10% glycerol, 2% Triton X-100, 1 mM phenylmethylsulfonyl fluoride, and protease inhibitor cocktail (Calbiochem, USA) and quantified using the Bradford assay (Pierce, USA). Equal quantities of protein were electrophoresed in a 10% SDS-PAGE gel and transferred to PVDF membrane. The blots were probed with primary antibody goat anti human lumican at 1:1000 (R&D Systems, Minneapolis, USA) and with secondary antibody HRP-anti goat antibody at 1:5000 (R&D Systems, Minneapolis, USA). Immunocomplexes were visualized using an enhanced chemiluminiscent substrate (KPL, MN, USA). Following initial probing for lumican, the blots were stripped and reprobed for beta actin (Abeam, USA).

### Immunofluorescence Staining

HuH7 or LX2 cells were plated at uniform density in single well chamber slides adopting the same protocol as for cell culture described above. At time points of 0, 24, 48, and 72 hours post seeding, medium was aspirated, slides were washed with phosphate buffered saline, air dried and stored at −80° C. until staining. Briefly, cells were fixed in acetone, followed by 1% paraformaldehyde. Cells were immunostained with anti human lumican at dilution of 1:50 (Santa Cruz, USA) and detected with appropriate FITC labeled secondary antibodies (MP Biomedicals, OH, USA). Negative controls consisted of identical treatments with omission of primary antibody. DAPI was used for nuclear staining (Sigma, MO, USA). Images were captured on an Olympus microscope with appropriate filter settings for FITC (green) and DAPI (blue).

### Immunohistochemistry

Formalin preserved paraffin embedded liver biopsy sections collected from patients with chronic HCV patients (n=6) and also from rodent (J129/B6 mice) liver that were administered carbon tetrachloride twice weekly at 1 mL/kg for one month (n=6) were used for detecting presence of lumican. Deparaffinized, hydrated human and mouse liver tissue sections were stained with primary antibody against human lumican (R & D, USA) at a dilution of 1:1000. Bound antibodies were detected using diaminobenzoid and sections were counter stained with hematoxylin.

### Statistical Analysis

Gene expression data presented as copy number are expressed as mean±standard error. Fold changes represent values normalized to 18S and to time point of 0 hours. To assess significance of difference between treatments, the paired 2-tailed student's test with equal variance was used. A p value of <0.05 was considered statistically significant.

### Results

### Lumican Expression is Upregulated in Chronic Liver Injury

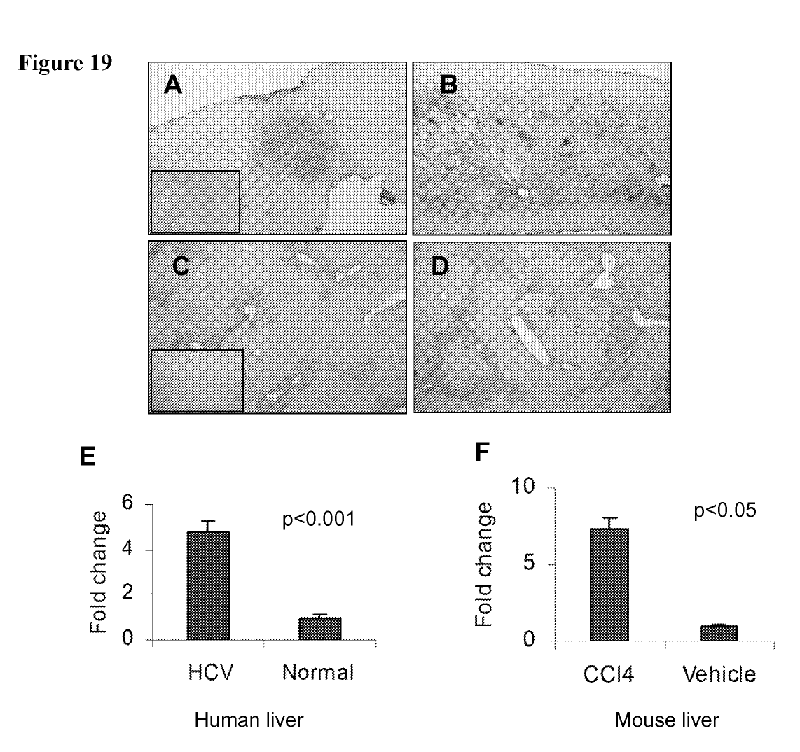

In order to evaluate whether lumican over expression was a generalized response to chronic liver injury, the presence of lumican was examined in immunostained liver biopsy samples obtained from patients with chronic hepatitis C infection. Immunohistochemical analysis was performed on liver tissue sections of mice subjected to chronic carbon tetrachloride injury at 1 mL/kg twice weekly for four weeks. In biopsy sections of HCV infected patients; lumican was present within the sinusoids and within the cytoplasm of hepatocytes in an irregular mosaic pattern across the zones (FIGS. 19A and 19B) as compared to normal (FIG. 19A, inset). In mice administered carbon tetrachloride, lumican was over expressed in sinusoids and within the cytoplasm of hepatocytes clustered around the areas of inflammation and fibrosis (FIGS. 19C and 19D) as compared to its minimal and generalized distribution in mice administered vehicle only (FIG. 19C, inset). Gene expression was evaluated in both groups and was found to be increased fivefold (p<0.01) in HCV infected liver tissue when compared to normal liver (FIG. 19E) and likewise significantly increased seven fold in carbon tetrachloride injured mouse liver as compared to normal mouse liver (FIG. 19F).

### Lumican Expression in Cells of Differing Hepatic Origin

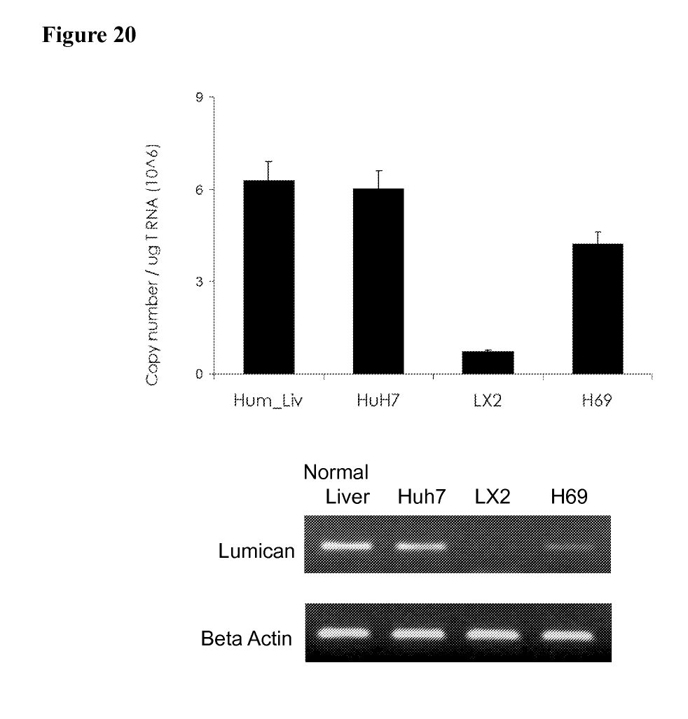

Expression of lumican was compared in three cell lines of differing hepatic origin to evaluate their likely differential contribution to lumican expression within the sinusoids of the liver. There was an approximately eight fold statistically significant increase in lumican expression in the HuH7 hepatocyte cell line and in normal liver samples as compared to the hepatic stellate cell line LX2 (p<0.05). There was also a 1.5 fold greater contribution of lumican from the cholangiocytes as compared to the stellate cells (FIG. 20). Lumican expression in HuH7 cells was comparable to that of normal liver (p>0.05).

Response of Lumican Expression to Free Fatty Acids (FFA)

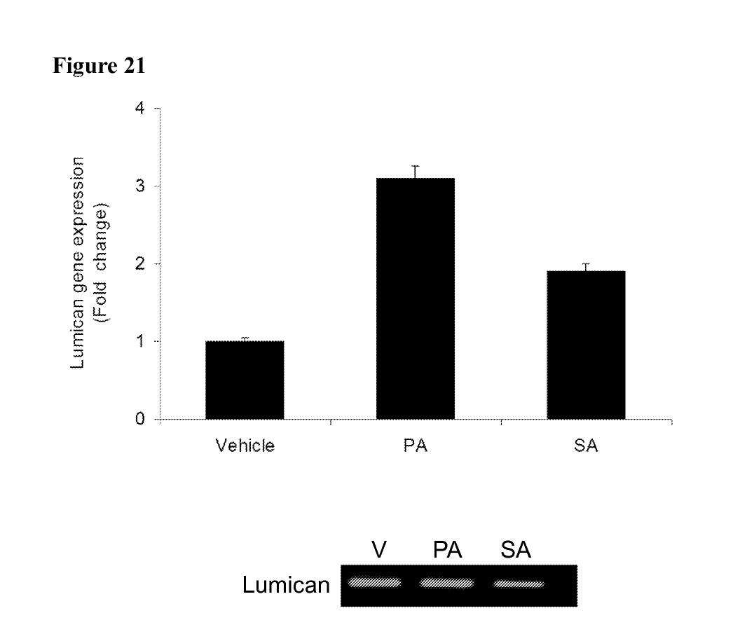

In response to short term exposure of free fatty acids (palmitic acid and stearic acid) similar to fasting FFA plasma concentration observed in human non alcoholic steatohepatitis (Malhi et al., J. Biol. Chem., 281:12093-12101 (2006)), there was an increase in lumican expression in HuH7 cells. Exposure to palmitic acid was associated with a 3.1 fold increase in lumican expression as compared to the 1.9 fold change when exposed to stearic acid (p=0.05) (FIG. 21).

### Response of HuH7 and LX2 Cells to TGFβ1

### Lumican Expression

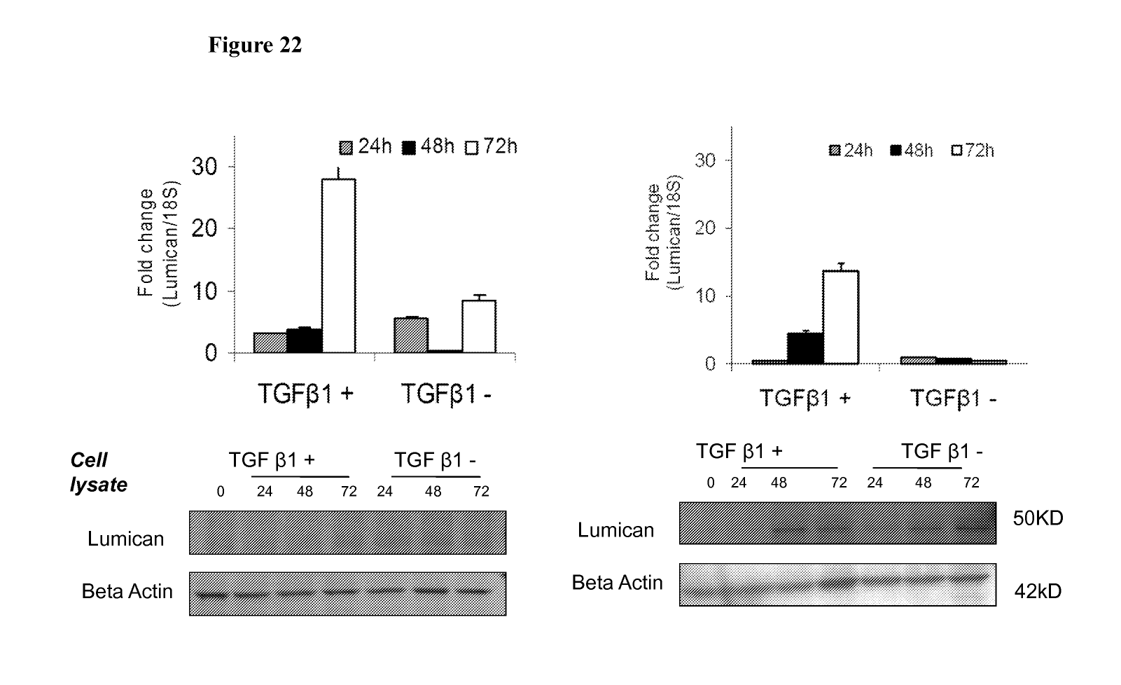

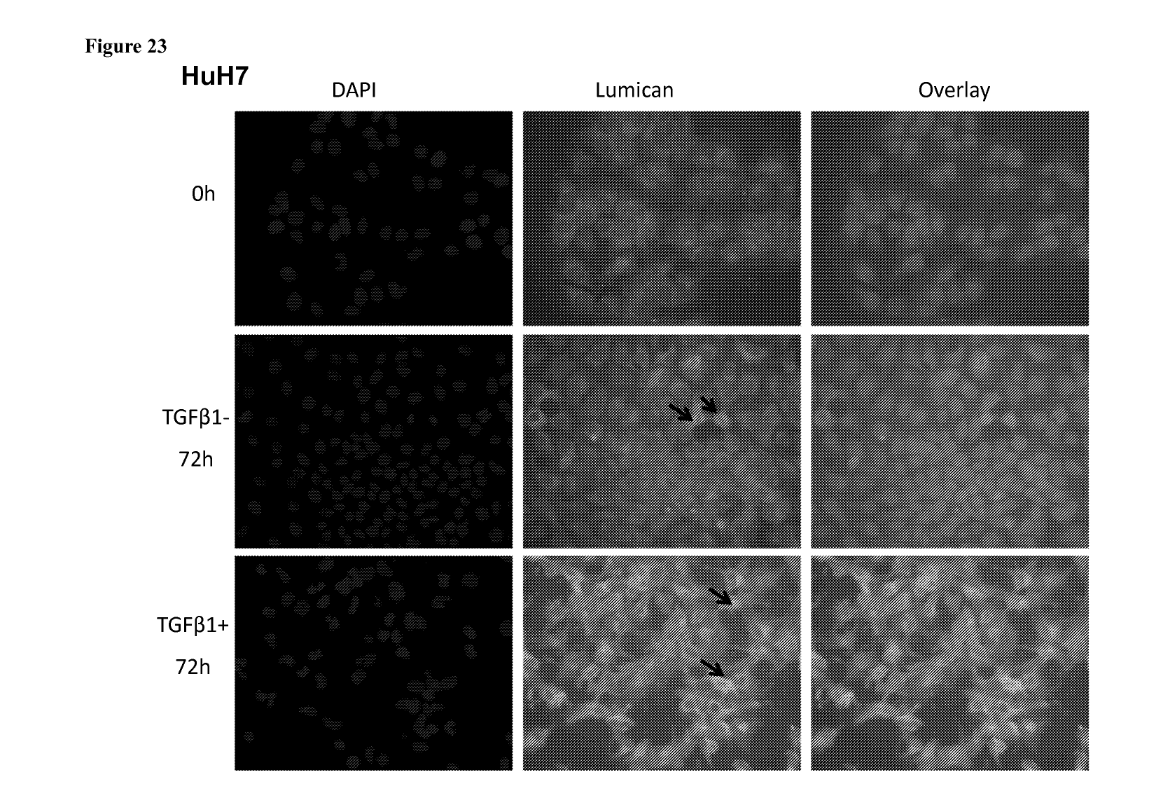

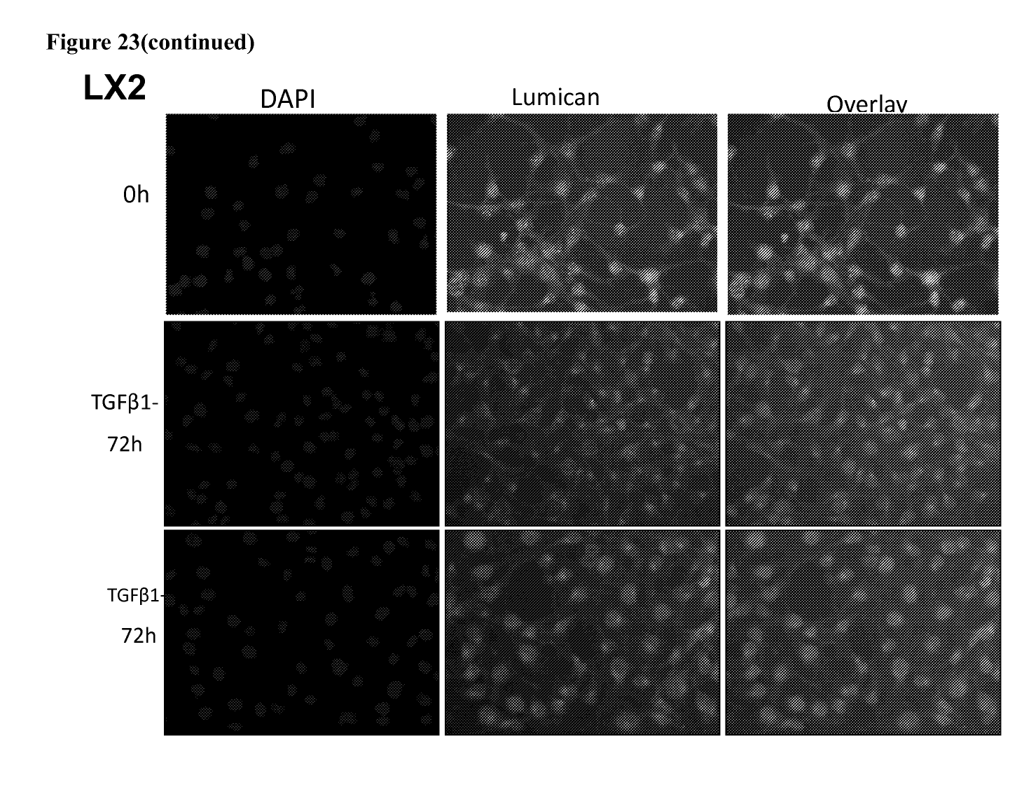

The effect of the pro-fibrotic cytokine TGFβ1 at 2 ng/mL on the gene expression of lumican was examined in HuH7 as well as LX2 cell lines over a period of 72 hours. The results demonstrated that the epithelial hepatocytes and mesenchymal stellate cells responded differently to TGFβ1 signaling. Over a period of 72 hours, hepatocytes underwent apoptosis as indicated by decreasing cell numbers (FIG. 22) and by observations of floating dead cells in the culture medium at all time points. This could also be observed in DAPI stained nuclei of HuH7 cells using immunofluorescent staining for lumican at 72 hours (FIG. 23). The stellate cells continued to proliferate at the same rate as untreated cells (FIG. 22).

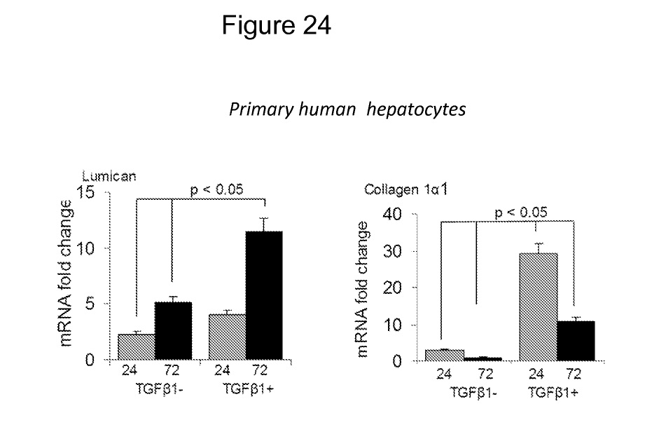

The cytokine TGFβ1 caused an increase in lumican gene expression in both hepatocytes as well as stellate cells. In terms of fold change this effect was more profound in stellate cells at the 72 hour time period (27 fold difference over baseline; p<0.05) than in hepatocytes (a 15 fold increase over baseline; p<0.05). Similar increases in gene expression were observed for primary human hepatocytes (FIG. 24). Untreated stellate cells showed a marginal stepwise increase in lumican expression over the 72 hour time period as compared to no changes observed for the hepatocytes (FIG. 22). The protein expression of lumican was examined in HuH7 cells and in LX2 cells (FIG. 23) over 72 hours using immunofluorescent assays. In both the hepatocyte cell line as well as the stellate cells, lumican expression is visible that was greatly enhanced at 72 hours in the HuH7 cells exposed to TGFβ1. DAPI stained nuclei of HuH7 cells appeared apoptotic and fragmented (FIG. 23).

Western blot analysis of cell lysate and the supernatants revealed two bands of differing molecular weights, 110 kD and 50 kD in both HuH7 and LX2 supernatants. The 110 kD band predominated in the LX2 cell lysate while the 50 kD occurred in the HuH7 cell lysates (FIG. 22).

### Collagen 1 α1 Expression

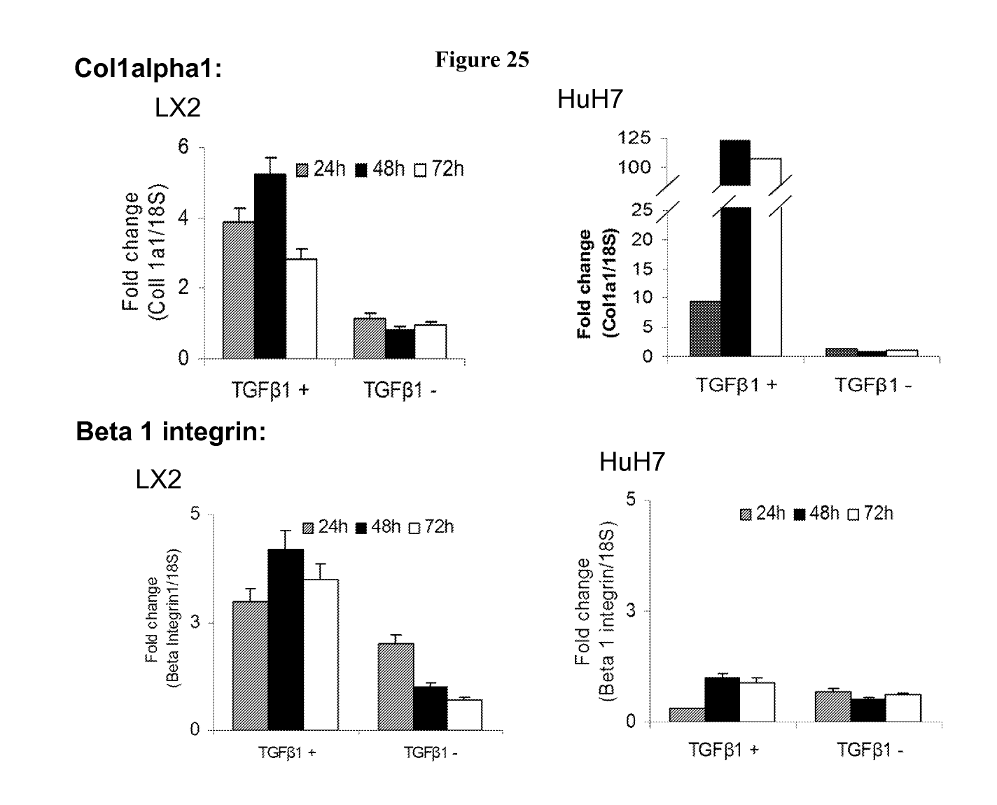

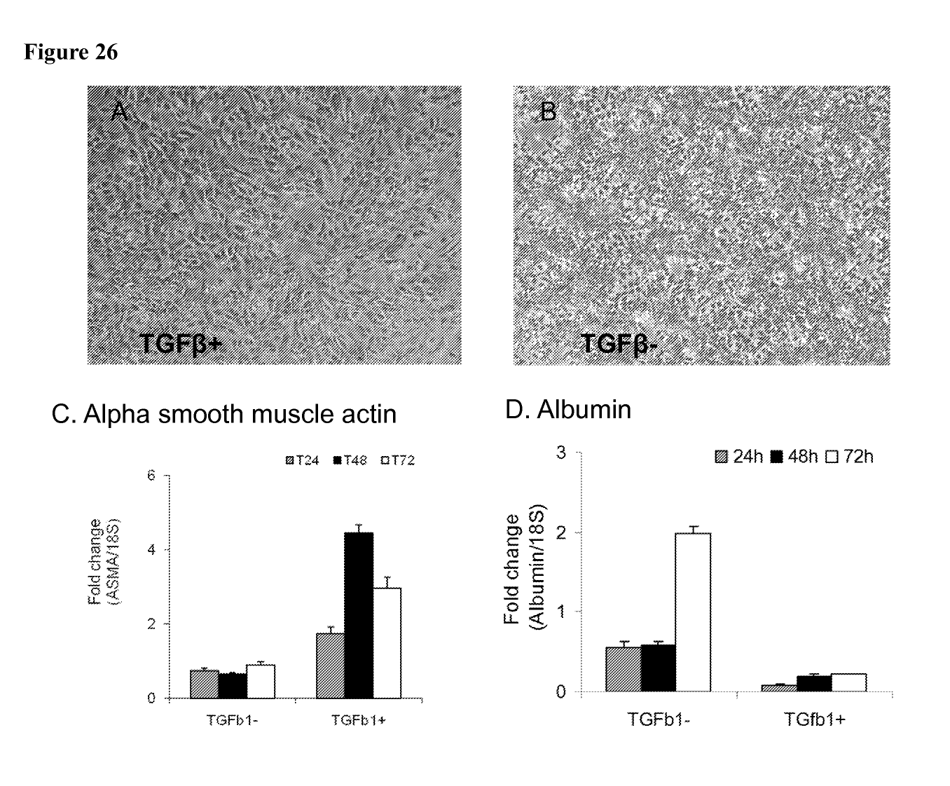

There was no increase in collagen 1α1 gene expression in untreated cells for both cell lines (FIG. 25). In contrast, in TGFβ1 treated cells, there was a gradual increase in expression of collagen 1α1 measuring as much as 9-fold by 24 hours and increasing to >100 fold by 48 hours in the Huh? cells. Similar results were observed in primary human hepatocytes treated with TGFβ1 (FIG. 24). For stellate cells, there was an initial 4 fold increase by 24 hours that was further enhanced to a 5 fold increase by 48 hours (p<0.05).

### β1 Integrin Expression

The expression of the known cellular receptor of lumican, beta 1 integrin, was measured in both HuH7 and LX2 cells. In cells treated with TGFβ1, there was gradual increase in expression of beta 1 integrin only in the stellate cells, while no change was observed for beta 1 integrin expression in hepatocytes (FIG. 25).

### Alpha Smooth Muscle Actin and Albumin Expression

The expression of markers of liver epithelial cells, albumin, and a marker of stellate cell activation alpha smooth muscle actin were measured in HuH7 cells to determine whether the epithelial cells were undergoing a transition to a mesenchymal phenotype. By 48 hours, there was a fourfold increase in alpha smooth muscle expression. On the other hand, albumin production was decreased 10 fold by 24 hours and leveled to a five fold decrease by 48-72 hours (FIG. 23). In the LX2 cells, there was a gradual increase in a smooth muscle actin measuring a 4.5 fold increase by 48 hours (FIG. 23). By contrast, there was no increase in alpha smooth muscle actin in the LX2 cells (FIG. 23).

### Example 4

### Lumican is Upregulated in HCV Infection and In Other Models of Chronic Liver Injury

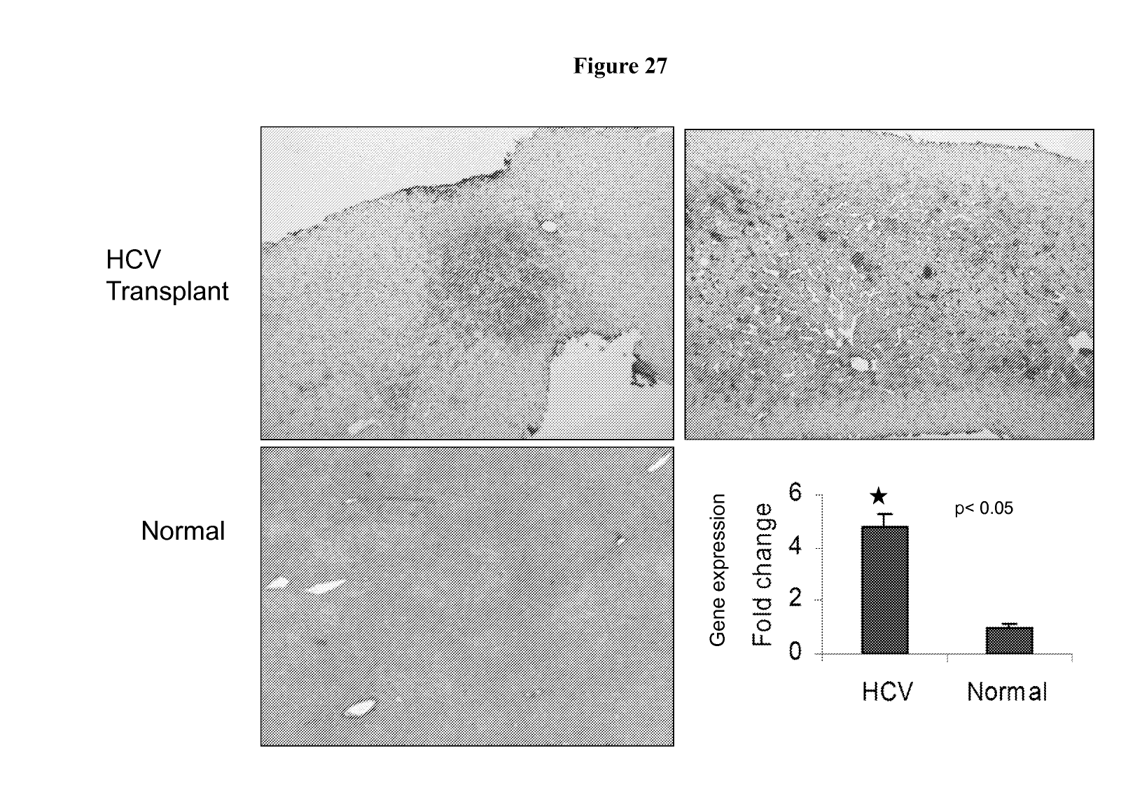

Liver tissue samples were obtained from transplant patients with hepatitis C virus (HCV) and normal patients without liver disease. Staining for lumican expression in samples showed that lumican expression is upregulated in the tissue from transplant patients when compared to normal liver tissue samples (FIG. 27). Lumican gene expression was also examined from these tissues and showed a marked increase in lumican gene expression in HCV transplant patients versus normal tissue samples (FIG. 27).

These results demonstrate that lumican may be involved in a common pathway to hepatic fibrosis.

### Example 5

### In Vitro Assay to Identify an Inhibitor of Lumican Lumican Abundance is Measured in Tissue or Serum using Anti-Lumican Antibodies (ELISA for Serum or Immunohistochemistry for Tissue)

In brief, biopsy sections are deparaffinized successively, hydrated in deionized water, and washed in buffer (DAKO, USA). After blocking endogenous peroxidase (DAKO S2001, DAKO, Carpenteria, Calif., USA), sections are washed with buffer (DAKO S3006; Tris-buffered saline containing 0.05% Tween, pH7.6) and background blocked for 5 minutes (SNIPER, Biocare Medical, Concord, Calif., USA). Sections are then incubated with primary goat anti-human lumican at 1:1000 (R&D systems, Minneapolis, Minn., USA) in a background reducing diluent for 1 hour at room temperature. After washing (DAKO S3006), the tissue is incubated with horseradish peroxidase-labeled anti-goat antibody (Promark Goat Polymer, Biocare Medical). After further washings, sections are developed with betazoid diamobenzidine (Biocare Medical) for 10 minutes at room temperature and then counterstained with hematoxylin for 5 minutes. For negative controls, tissue sections are incubated without primary antibody in TBS and 1% BSA.

For serum, ELISA assays for lumican based on commercially available anti-lumican is measured in the serum of patients suspected liver disease. Abundance of lumican measured by ELISA is calculated using spline-algorithm and expressed as ng/mL. Optical density is measured within 30 minutes at 450 nm.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having a liver fibrosis condition, wherein said method comprises administering, to said mammal, an inhibitor of lumican under conditions wherein the severity of said liver fibrosis condition is reduced.

2. The method of claim 1, wherein said mammal is a human.

3. The method of claim 1, wherein said method comprises identifying said mammal as having said liver fibrosis condition prior to said administering step.

4. The method of claim 1, wherein said method comprises identifying said mammal as having said liver fibrosis condition and as being in need of said administration.

5. The method of claim 1, wherein said method comprises assessing said mammal, after said administering step, for a reduction in said severity.

6. The method of claim 1, wherein said inhibitor is an anti-lumican antibody.

7. The method of claim 1, wherein said inhibitor is an siRNA directed against a nucleic acid encoding a lumican polypeptide.

8. A method for identifying a treatment agent for treating a liver fibrosis condition, wherein said method comprises:
(a) determining whether or not a test agent inhibits lumican expression or activity, wherein inhibition of lumican expression or activity indicates that said test agent is a candidate agent, and
(b) administering said candidate agent to a mammal having said liver fibrosis condition to determine whether or not said candidate agent reduces the severity of said liver fibrosis condition, wherein a reduction in said severity indicates that said candidate agent is said treatment agent.

9. The method of claim 8, wherein said step (a) comprises using an in vitro lumican expression assay.

10. The method of claim 8, wherein said mammal is a mouse.

11. A method for treating a mammal suspected to develop said liver fibrosis condition, wherein said method comprises administering, to said mammal, an inhibitor of lumican under conditions wherein development of said liver fibrosis condition is reduced.

12. The method of claim 11, wherein said mammal is a human.

13. The method of claim 11, wherein said method comprises identifying said mammal as being likely to develop said liver fibrosis condition prior to said administering step.

14. The method of claim 11, wherein said method comprises identifying said mammal as being likely to develop said liver fibrosis condition and as being in need of said administration.

15. The method of claim 11, wherein said method comprises assessing said mammal, after said administering step, for a reduction in said development.

16. The method of claim 11, wherein said inhibitor is an anti-lumican antibody.

17. The method of claim 11, wherein said inhibitor is an siRNA directed against a nucleic acid encoding a lumican polypeptide.

18. A method of identifying a mammal in need of treatment with an inhibitor of lumican, wherein said method comprises detecting the presence of an elevated level of lumican polypeptide in a mammal, and classifying said mammal as being in need of treatment with an inhibitor of lumican based at least in part on the presence of said elevated level.

19. The method of claim 18, wherein said mammal is a human.

20. The method of claim 18, wherein said mammal is a mammal suspected of having liver fibrosis.


SEQUENCE LISTING (20 sequences)

SEQ ID NO: 1 - DNA, 23 bp, Homo sapiens
cttcaatcagatagccagactgc

SEQ ID NO: 2 - DNA, 23 bp, Homo sapiens
agccagttcgttgtgagataaac

SEQ ID NO: 3 - DNA, 20 bp, Mus musculus
tcgagcttgatctctcctat

SEQ ID NO: 4 - DNA, 21 bp, Mus musculus
tggtcccaggatcttacagaa

SEQ ID NO: 5 - DNA, 20 bp, Homo sapiens
ctcaacacgggaaacctcac

SEQ ID NO: 6 - DNA, 20 bp, Homo sapiens
cgctccaccaactaagaacg

SEQ ID NO: 7 - DNA, 19 bp, Homo sapiens
ggtaacagcggtgaacctg

SEQ ID NO: 8 - DNA, 19 bp, Homo sapiens
gagctcctcgctttccttc

SEQ ID NO: 9 - DNA, 20 bp, Mus musculus
ctcctggcaagaatggagat

SEQ ID NO: 10 - DNA, 18 bp, Mus musculus
aatccacgagcaccctga

SEQ ID NO: 11 - DNA, 23 bp, Mus musculus
ggaacaccttaagtcactggaca

SEQ ID NO: 12 - DNA, 20 bp, Mus musculus
tgggttcttggctgtgtttt

SEQ ID NO: 13 - DNA, 21 bp, Mus musculus
tgcctatgtctcagcctcttc

SEQ ID NO: 14 - DNA, 20 bp, Mus musculus
gaggccatttgggaacttct

SEQ ID NO: 15 - DNA, 20 bp, Mus musculus
tggagcaacatgtggaactc

SEQ ID NO: 16 - DNA, 18 bp, Mus musculus
cagcagccggttaccaag

SEQ ID NO: 17 - DNA, 23 bp, Mus musculus
accagtctccgaggagaaactat

SEQ ID NO: 18 - DNA, 23 bp, Mus musculus
ggactttgtcaaaaagagctcag

SEQ ID NO: 19 - DNA, 22 bp, Mus musculus
tggagggcctggagaacctcac

SEQ ID NO: 20 - DNA, 23 bp, Mus musculus
gtgcagaagctgctgatggagaa